(19) United States
(12) Patent Application Publication
(10) Pub. No.: US 2024/0325147 A1
(43) Pub. Date: Oct. 3, 2024

(54) HEART VALVE PROSTHESIS DELIVERY SYSTEM

(71) Applicant: JC Medical, Inc., Burlingame, CA (US)

(72) Inventors: Brandon G. WALSH, Burnaby (CA); Ji ZHANG, Burnaby (CA); Alex Thorndike ROTH, Redwood City, CA (US); Jinhua ZHU, San Francisco, CA (US)

(21) Appl. No.: 18/622,807

(22) Filed: Mar. 29, 2024

Related U.S. Application Data
(60) Provisional application No. 63/455,943, filed on Mar. 30, 2023.

(51) Int. Cl. A61F 2/24; A61F 2/95

(52) U.S. Cl. CPC A61F 2/2436; A61F 2/2412; A61F 2/9522

(57) ABSTRACT

A prosthetic heart valve delivery device can include an engagement mechanism that can securely engage with and reliably release a heart valve prosthesis carried thereby. The delivery device can have a docking area defining a longitudinal axis and an engagement wire extending along the longitudinal axis. The engagement wire can have an engaged position in which the engagement wire extends through the docking area to engage with the heart valve prosthesis. The engagement wire can also be moved to a disengaged position to permit disengagement of the heart valve prosthesis from the docking area.

### CROSS-REFERENCE TO RELATED APPLICATIONS

The present application claims priority to and the benefit of U.S. Provisional App. No. 63/455,943, filed on Mar. 30, 2023, which is related to International Application No. PCT/US2019/012406, filed on Jan. 4, 2019, and International Application No. PCT/US2019/012408, filed on Jan. 4, 2019, the entireties of each of which are incorporated herein by reference.

### TECHNICAL FIELD

The present disclosure relates to devices, systems, and methods for the percutaneous delivery and implantation of a cardiac valve prosthesis. The valve delivery device can securely retain can be delivered in a compressed state within a sheath to the defective native valve and released in situ.

### BACKGROUND

Prosthetic heart valves are used to replace damaged or diseased heart valves. In vertebrate animals, the heart is a muscular organ with four pumping chambers: the left and right atria and the left and right ventricles, each provided with its own one-way valve. The natural heart valves are identified as the aortic, mitral (or bicuspid), tricuspid and pulmonary valves. Prosthetic heart valves can be used to replace any of these naturally occurring valves, although repair or replacement of the aortic or mitral valves is more common since they reside in the left side of the heart where pressures are the greatest.

A conventional heart valve replacement surgery involves accessing the heart in the patient's thoracic cavity through a longitudinal incision in the chest. For example, a median sternotomy requires cutting through the sternum and forcing the two opposing halves of the rib cage to be spread apart, allowing access to the thoracic cavity and heart within. The patient is then placed on cardiopulmonary bypass which involves stopping the heart to permit access to the internal chambers. Such open-heart surgery is particularly invasive and involves a lengthy and difficult recovery period.

The foregoing examples of the related art and limitations related therewith are intended to be illustrative and not exclusive. Other limitations of the related art will become apparent to those of skill in the art upon a reading of the specification and a study of the drawings.

### SUMMARY

The present disclosure relates to heart valve prostheses, delivery devices, and actuation handles that can facilitate delivery of a heart valve prosthesis to a defective native valve structure in a patient, such as the aortic valve. In some embodiments, the delivery can be performed using a transcatheter approach.

The delivery devices disclosed herein can enable a clinician to more easily deliver and disengage from a heart valve prosthesis carried thereby. The delivery device can advance through blood vessels leading to the heart, as well as through tortuosities of such vessels, using a transvascular approach, such as a transfemoral approach. In accordance with an aspect of at least some embodiments disclosed herein is the realization that in advancing a heart valve prosthesis to a target location within the heart, a delivery device must provide a secure engagement between the delivery device and the heart valve prosthesis; however, the delivery device must also be able to reliably disengage from and release the heart valve prosthesis at the target location without any trauma to the native tissue or damage to the heart valve prosthesis itself. Further, this realization also notes that few delivery devices have successfully balanced these competing needs. Therefore, the present disclosure addresses these and other issues and provides significant advances that ensure safety and reliability in operating a heart valve delivery device.

A prosthetic heart valve delivery device can include an engagement mechanism that can securely engage with and reliably release a heart valve prosthesis carried thereby. The delivery device can have a docking area defining a longitudinal axis and an engagement wire extending along the longitudinal axis. The engagement wire can have an engaged position in which the engagement wire extends through the docking area to engage with the heart valve prosthesis. The engagement wire can also be moved to a disengaged position to permit disengagement of the heart valve prosthesis from the docking area.

In accordance with some embodiments, procedures are provided for a transcatheter aortic valve implantation (TAVI) and/or a transcatheter aortic valve replacement (TAVR). For example, in the TAVI procedure, a clinician can anchor the valve anchor of the heart valve prosthesis relative to the aortic valve annulus to guide the placement of the prosthetic leaflet structure. The valve prosthesis can comprise prosthetic leaflets, a valve anchor, a valve frame component, and a tethering component, which allows the valve anchor and the frame component to be placed serially in a delivery device in order to reduce the overall crossing profile of the delivery device. In accordance with some embodiments, the valve anchor can be coupled to one or more grasper mechanisms of a delivery device. The grasper mechanism can be configured to include one of a variety of unique structures disclosed herein that both securely engage with a portion of the valve anchor and permit reliable release of the valve anchor therefrom.

Additional embodiments of the present devices and methods, and the like, will be apparent from the following description, drawings, examples, and claims. As can be appreciated from the foregoing and following description, each and every feature described herein, and each and every combination of two or more of such features, is included within the scope of the present disclosure provided that the features included in such a combination are not mutually inconsistent. In addition, any feature or combination of features may be specifically excluded or omitted from any embodiment of the present disclosure. Additional aspects and advantages of the present disclosure are set forth in the following description and claims, particularly when considered in conjunction with the accompanying examples and drawings.

Additional features and advantages of the subject technology will be set forth in the description below, and in part will be apparent from the description, or may be learned by practice of the subject technology. The advantages of the subject technology will be realized and attained by the structure particularly pointed out in the written description and embodiments hereof as well as the appended drawings.

Certain features of valve prostheses, delivery devices, actuation handles, other devices, systems, and methods which can be implemented with the valve prostheses, delivery devices, actuation handles, other devices, systems, and methods discussed in the present disclosure, can implement features of and/or be used in combination with other features of valve prostheses, delivery devices, actuation handles, other devices, systems, and methods described for example in International Application No. PCT/US2019/012406, filed on Jan. 4, 2019, and in International Application No. PCT/US2019/012408, the entirety of each of which is incorporated herein by reference.

It is to be understood that both the foregoing general description and the following detailed description are exemplary and explanatory and are intended to provide further explanation of the subject technology.

### BRIEF DESCRIPTION OF THE DRAWINGS

Various features of illustrative embodiments of the inventions are described below with reference to the drawings. The illustrated embodiments are intended to illustrate, but not to limit, the inventions. The drawings contain the following figures:

FIG. 1 illustrates delivery of a valve prosthesis using a valve delivery device in a transfemoral retrograde approach, according to some embodiments.

FIG. 2 shows a valve prosthesis, according to some embodiments.

FIG. 3 is a partial, side cross-sectional view of the valve prosthesis of FIG. 2 loaded onto a valve delivery device, according to some embodiments.

FIG. 4 is a perspective view the valve delivery device of FIG. 2 showing a grasper mechanism in engagement with a valve anchor and in an enlarged state, according to some embodiments.

FIG. 5A is an enlarged perspective view the valve delivery device of FIG. 3, showing the grasper mechanism for engaging the valve anchor, according to some embodiments.

FIG. 5B is an enlarged perspective view the valve delivery device of FIG. 3, showing a tubular member of the grasper mechanism in dashed lines to illustrate internal components of the grasper mechanism, according to some embodiments.

FIGS. 6A and 6B is a cross-sectional view of the valve delivery device of FIG. 5A, according to some embodiments.

FIGS. 7A-7D illustrate a disengagement process of the valve delivery device of FIG. 5A, according to some embodiments.

FIG. 8 illustrates an alternative grasper mechanism in an engaged state, according to some embodiments.

FIG. 9 illustrates another grasper mechanism, in an engaged state, according to some embodiments.

FIG. 10 illustrates yet another grasper mechanism, in an engaged state, according to some embodiments.

FIG. 11 illustrates yet another grasper mechanism, according to some embodiments.

FIGS. 12A-12E illustrates yet another grasper mechanism and the disengagement process thereof, according to some embodiments.

FIG. 13 illustrates yet another grasper mechanism in a disengaged state, according to some embodiments.

FIG. 14 illustrates yet another grasper mechanism in an engaged state, according to some embodiments.

FIGS. 15A and 15B illustrate yet another grasper mechanism and the disengagement process thereof, according to some embodiments.

FIGS. 16A and 16B illustrate yet another grasper mechanism and the disengagement process thereof, according to some embodiments.

FIG. 17 illustrates yet another grasper mechanism and the disengagement process thereof, according to some embodiments.

### DETAILED DESCRIPTION

In the following detailed description, numerous specific details are set forth to provide a full understanding of the subject technology. It should be understood that the subject technology may be practiced without some of these specific details. In other instances, well-known structures and techniques have not been shown in detail so as not to obscure the subject technology.

Further, while the present disclosure sets forth specific details of various embodiments, it will be appreciated that the description is illustrative only and should not be construed in any way as limiting. Additionally, it is contemplated that although particular embodiments of the present disclosure may be disclosed or shown in the context of aortic valve prostheses, such embodiments may be used in other cardiac valve prosthesis applications. Furthermore, various applications of such embodiments and modifications thereto, which may occur to those who are skilled in the art, are also encompassed by the general concepts described herein.

Various embodiments will now be described more fully hereinafter. Such embodiments may, however, be embodied in many different forms and should not be construed as limited to the embodiments set forth herein; rather, these embodiments are provided so that this disclosure will be thorough and complete, and will fully convey its scope to those skilled in the art. Thus, one or more features shown or otherwise disclosed in an embodiment herein may be interchangeably used or incorporated into another embodiment that may not expressly show or disclose such feature(s). Further, one or more features shown or otherwise disclosed for an embodiment herein may be excluded from such embodiment, unless expressly indicated, using skill in the art.

As with all cardiac valves, a healthy aortic valve will open to allow blood flow and close to prevent backflow of blood. However, disease and dysfunction of the valve can result in regurgitation or decreased blood flow (stenosis). In such cases, a replacement aortic valve prosthesis must be used to perform the functions of a healthy aortic valve.

Minimally invasive surgical techniques are evolving, where a valve prosthesis can be introduced into a patient using a catheter that is introduced via a small incision that provides access to, for example, a femoral artery or directly to the heart. These implantation techniques have shown promising results in providing treatment options for patients who are poor open surgical candidates. Nevertheless, challenges still remain in such catheter-based delivery of prosthetic valves.

For example, in according with an aspect of at least one embodiment disclosed herein is the realization that advancing a conventional tubular delivery device through a vessel exerts stress against the vessel walls and carries the risk of damaging the vessel walls. Further, in according with an aspect of at least one embodiment disclosed herein is the realization that transcatheter prosthetic valves may not be able to treat patients with aortic regurgitation. Additionally, in according with an aspect of at least one embodiment disclosed herein is the realization that conventional prosthetic valves may be difficult to position, may require rapid ventricular pacing, and may have limited expansion. Accordingly, implantation and use of conventional prosthetic valves may result in complications, such as vascular damage, moderate to severe paravalvular leakage, valve thrombosis/migration, coronary artery blockage, and excessive stress due to excessive radial force.

The present disclosure describes various aspects of heart valve prostheses that can be delivered to a defective heart valve in a patient. The valve prostheses can comprise at least one valve anchor, which is movably connected, movably attached, flexibly connected, displaceably connected, linked, or coupled to a radially expandable valve support or frame. The valve frame can comprise prosthetic valve leaflets or cusps and provide the functionality of the native heart valve. Certain features of valve prostheses, which can be implemented with the prostheses discussed in the present disclosure, are also further described for example, in U.S. Pat. No. 8,366,768, the entirety of which is incorporated herein by reference.

Thus, the present disclosure provides a variety of features that can be optionally incorporated or excluded from any of the embodiments explicitly discussed or illustrated herein. These modifications and combinations of features can be performed by a person of skill to achieve advantages and benefits discussed herein. Further, certain modifications or combinations are indicated or suggested herein, but it is contemplated that a person of skill in the art can implement or exclude certain aspects or features disclosed herein in developing a suitable embodiment or implementation of these teachings. Advantageously, various embodiments described herein allow for treating patients with aortic regurgitation, permit precise axial, angular, and radial positioning of the valve prosthesis, minimize valve migration and paravalvular leakage while avoiding damage to the valve annulus, minimize the need for a pacemaker, and decrease the likelihood of blocking the coronary artery.

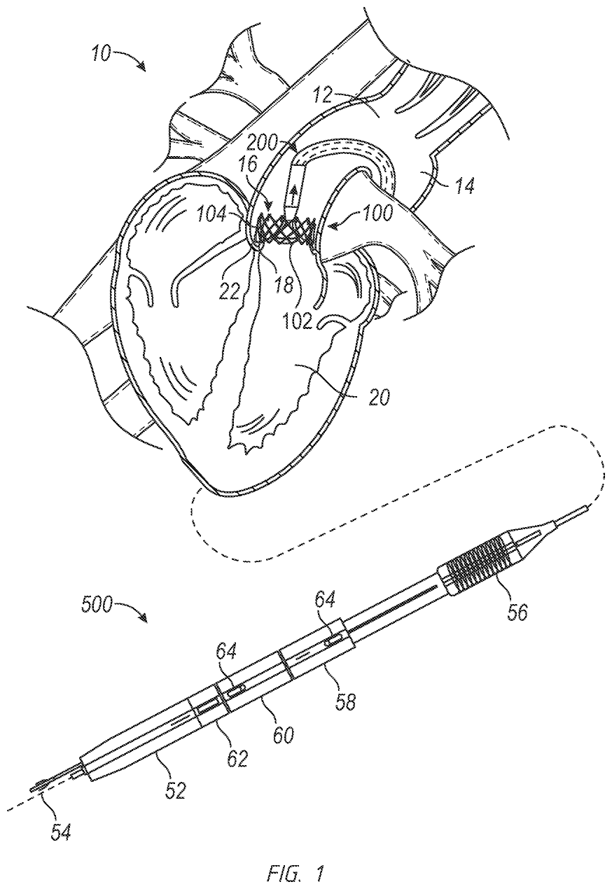

Some of these features and benefits of a heart valve prosthesis and a delivery system thereof are illustrated with respect to FIGS. 1-4. FIG. 1 illustrates the use of the delivery device in a human heart 10. The heart 10 can comprise an aorta 12 having an aortic arch 14 and an aortic valve 16. The aorta valve 16 can comprise a plurality of native valve leaflets 18 and separate the aorta 12 from the left ventricle 20. In accordance with some embodiments, a clinician can operate a handle actuator 50 to control the delivery and release of a valve prosthesis 100. The valve prosthesis 100 can be carried by a delivery device to be advanced retrograde through the aorta 12 until reaching and being positioned through the native valve leaflets 18 of the aortic valve 16.

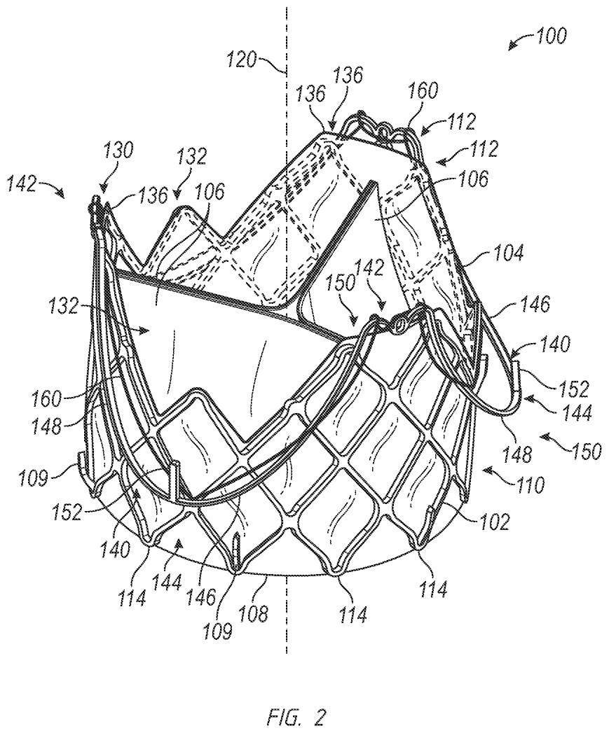

With reference to FIGS. 1 and 2, during delivery of the valve prosthesis 100 to the native valve site, a support frame 102 and a valve anchor 104 of the prosthesis 100 can be positioned in tandem, as an axially displaced unit (with or without partial or full overlapping between the anchor and the frame) along the longitudinal axis of the delivery device 200. This configuration, as opposed to a concentric arrangement, can allow a more radially compact configuration of the components of the valve prosthesis 100, creating a much smaller cross-section and facilitating a catheter-based delivery. This can improve the flexibility of the delivery device 200, enabling the delivery device 200 to be advanced over a guidewire through the tortuous geometries of the circulatory system, and in particular, the aortic arch 14. Indeed, even with guidewire-directed delivery devices, the aortic arch 14 represents a difficult obstacle due to its sudden and high-degree of curvature. Often, this is a limiting constraint for some surgeries or delivery devices. However, in accordance with the various benefits and advantages of some embodiments disclosed herein, as illustrated in FIG. 1, the delivery device 200 can be advanced over the aortic arch 14 to a target location in the region of the aortic valve 16.

As shown in FIG. 1, once the valve anchor 104 is in the desired position, the support frame 102 can be released from the distal carrier assembly and expanded into apposition with the native valve leaflets 18 and the internal aspects of the valve anchor 104, thus sandwiching the native valve leaflets 18 between the support frame 102 and the valve anchor 104. Advantageously, by sandwiching the native valve leaflets 18 between the support frame and the valve anchor, the valve prosthesis 100 can have reduced reliance on radial force retention. Further, by sandwiching the native valve leaflets 18 between the support frame and the valve anchor, the likelihood of the native valve leaflets 18 blocking the opening of the coronary artery is reduced, which may be beneficial for patients with low coronary ostia distance, and in patients with an existing valve prosthesis, who may need a new valve prosthesis inside the existing valve prosthesis (valve-in-valve application). The support frame and the valve anchor can thus expand into contact with the aortic valve 16, exerting a chronic outward force against the native valve leaflets 18 and aortic valve annulus 22. Thereafter, the prosthetic valve leaflets of the prosthesis 100 can begin to function in the manner desired and provide the same operation as a native valve.

According to some embodiments, the present disclosure also provides a handle actuator that can be used to control the operation of the presently disclosed delivery device and allow a clinician to reliably and accurately control the delivery of the valve prosthesis. FIG. 1 illustrates features and operation of the handle actuator 50 which can, according to some embodiments, be operated to deliver the valve prosthesis.

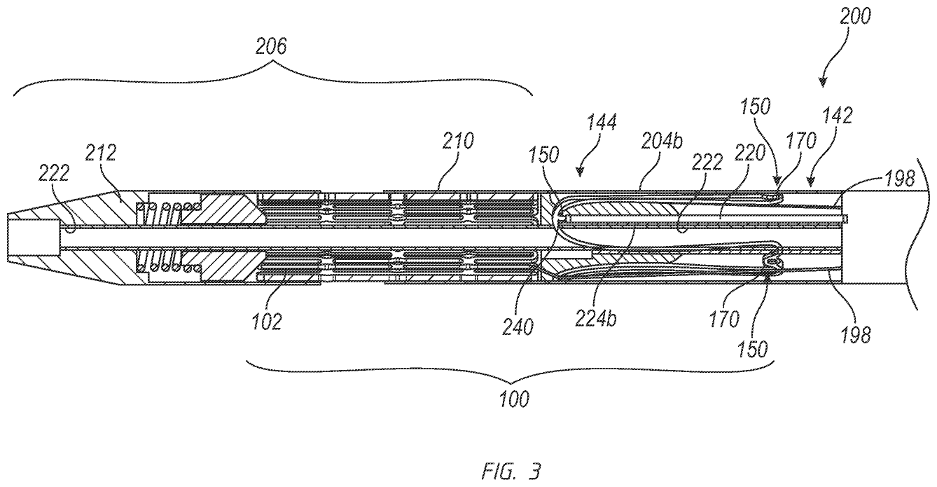

FIG. 1 illustrates the handle actuator 50, which can control one or more functions of a delivery device (e.g., the delivery device 200 discussed herein) for delivering of a valve prosthesis (e.g., the heart valve prosthesis 100 discussed herein). The handle actuator 50 can comprise a plurality of actuators or movable elements, such as knobs or buttons. The movable elements can permit a clinician to control one or more operations of the delivery device 200. The handle actuator 50 can comprise a control handle 52 having a longitudinal axis 54. The handle actuator 50 may be also referred to as a control unit. In some embodiments, the handle actuator 50 may be coupled to a second core member (shown, e.g., in FIG. 3). The control handle 52 can support the actuators and be held by the clinician during the procedure.

In some embodiments, as illustrated in FIG. 1, the handle actuator 50 can comprise a first movable element 56, a second movable element 58, a third movable element 60, and a fourth movable element 62. The first movable element 56 can be used to steer the delivery device 200, the second movable element 58 can be used to release the valve anchor, the third movable element 60 can be used to release nosecone or valve frame, and the fourth movable element 62 can be used as a nose cone toggle lock. The first movable element 56, the second movable element 58, the third movable element 60, and the fourth movable element 62 may be also referred to as the first control element 56, the second control element 58, the third control element 60, and the fourth control element 62.

Optionally, in some embodiments, one or more of the movable elements, such as the second movable element 58 and/or the third movable element 60, can include a button or slider safety switch 64 that prevent the unintentional rotation of the moveable elements. The safety switch 64 can be configured as resilient button or slider mechanisms that can be actuated to release a lock that provides resistance to rotational or translational movement of the respective movable element. In some embodiments, the movable elements can have a raised feature that provides a visual and tactile indication of rotational position such that the user does not need to look at the device in order to operate it, which can facilitate tactile engagement and actuation by the clinician. Other features of the handle actuator 50 and methods for operating the handle actuator 50 are discussed and illustrated in U.S. Pat. Nos. 11,090,156 and 11,083,577, the entireties of each of which is incorporated herein by reference.

Referring now to FIG. 2, a valve prosthesis 100 and components thereof are shown in various configurations. The valve prosthesis 100 can be delivered to a patient using a suitable delivery device, including embodiments of the delivery devices disclosed herein. The valve prosthesis 100 can comprise a support frame 102 and a valve anchor 104 to which the support frame 102 is movably connected, movably attached, flexibly connected, displaceably connected, linked, or coupled.

The valve prosthesis 100 can be configured such that components of the valve prosthesis 100 to be advanced in series while still being movably connected, movably attached, flexibly connected, displaceably connected, linked, or coupled to each other, thereby minimizing a passing profile or cross section of the delivery system. The interconnection of components of the valve prosthesis 100 can allow different degrees of motion and can be set into an engaged or retained position that provides a limited range of motion. In some embodiments, the engaged position can also provide a preset relative positioning of the components of the valve prosthesis 100 to facilitate proper placement and release of the valve prosthesis 100. Additionally, some embodiments can provide a clinician with a high degree of control and enhance the maneuverability of the valve prosthesis 100 when implanting the valve prosthesis 100 at the target location.

In some embodiments, the valve anchor 104 can be coupled to the support frame 102 when the support frame 102 is in the compact configuration prior to delivery and expansion. In some embodiments, the valve anchor 104 is not fixed to the support frame 102. Further, the valve anchor 104 can be separate from the support frame 102 or formed separately from and later coupled to the support frame 102. Thus, although at least a portion of the valve anchor 104, e.g., the anchoring leg, may be in contact with or otherwise reversibly attached or connected to the support frame 102, no part of the valve anchor 104 is fixed, e.g., welded or otherwise irreversibly adhered, to the support frame 102. Alternatively stated, the valve anchor 104, which may be in contact with or otherwise reversibly attached to the support frame 102, is not irreversibly fixed to the support frame 102.

Further, upon reaching the target location, the valve anchor 104 can be movably coupled to the support frame 102 in a manner that prevents the entire valve anchor 104 from being radially displaced from the support frame 102 when the valve anchor 104 is initially expanded. For example, portions of the valve anchor 104 can be radially displaced from the support frame during initial “landing” of the valve anchor 104 against the native valve structure at the target location. In some embodiments, the support frame 102 can be deployed or expanded within the native heart valve structure, and the valve anchor 104 can become sandwiched between the support frame and the native valve tissue, becoming at least partially, and possibly fully, immobilized. The valve anchor 104 can function to hold the expanded support frame 102 in place within the native valve structure.

Optionally, the support frame 102 may be referred to as a valve frame or valve support frame. FIG. 2 illustrates the support frame 102 aligned with and expanded within the valve anchor 104, in a configuration that is achieved when the prosthesis 100 is released and expanded within the native valve structure. The native valve structure includes the valve annulus or leaflets. This expanded configuration, serves to secure the valve prosthesis 100 within the native valve annulus by engaging the native valve structure. In some embodiments, the expanded configuration of the valve prosthesis 100 may reduce reliance on securing the valve prosthesis 100 with radial force exerted by the support frame 102 and the valve anchor 104 via the sandwiching or compression of the native valve leaflets between the support frame 102 and the valve anchor 104 of the valve prosthesis 100. Further, as discussed further herein, during implantation of the valve prosthesis 100, the support frame 102 and the valve anchor 104 can be movable relative to each other in expanded and/or compressed states in order to facilitate proper positioning of the prosthesis 100 relative to the native valve annulus and surrounding structures. Indeed, various advantages made possible by the prosthesis 100 delivery device disclosed herein allow a clinician to achieve a higher degree of precision in placing the prosthesis 100, as well as making such increased precision easier to achieve.

Referring to FIG. 2, the support frame 102 can comprise an outer or external surface and defines a central orifice about a longitudinal axis 120. The longitudinal axis 120 corresponds to an inflow-outflow axis of the prosthesis 100. In some embodiments, the valve prosthesis 100 further comprises a plurality of prosthetic valve leaflets or cusps 106 that are coupled to the support frame 102. The support frame 102 can provide a structural support for the valve leaflets 106. The valve leaflets 106 can have surfaces defining a reversibly scalable opening for unidirectional flow of a liquid through the prosthesis 100. The prosthesis 100 can include three valve leaflets 106 for a tri-leaflet configuration. As appreciated, mono-leaflet, bi-leaflet, and/or multi-leaflet configurations are also possible. For example, the valve leaflets can be coupled to the support frame 102 to span and control fluid flow through the lumen of the prosthesis 100. The prosthetic leaflets 106 can comprise one or more synthetic materials, engineered biological tissues, biological valvular leaflet tissues, pericardial tissues, cross-linked pericardial tissues, aortic root tissue, chemically or biologically processed/treated tissue, or combinations thereof. In some embodiments, the pericardial tissue is selected from but not limited to the group consisting of bovine, equine, porcine, ovine, human tissue, or combinations thereof.

Furthermore, in some embodiments, the valve prosthesis 100 can comprise a sealing component or membrane 108 that can be attached to an inside surface, an outside surface, and/or enclose the support frame 102, such as by being laminated onto inner and outer surfaces of the support frame 102. Thus, the valve leaflets 106 can be coupled to the support frame 102 and/or the membrane 108. In some embodiments, the membrane 108 can restrict blood flow in areas around the valve leaflets 106 so that blood flow occurs only between the valve leaflets 106 through the lumen of the prosthesis 100, as in a healthy native heart valve.

The support frame 102 and/or the valve anchor 104 can comprise a braided frame, a wire frame, or a laser-cut frame (e.g., laser-cut tubular mesh), as shown in FIG. 2. In some embodiments, the support frame 102 and/or the valve anchor 104 can comprise a shape-memory metal, which can change shape at a designated temperature or temperature range or by inducing stress. Alternatively, the self-expanding frames can include those having a spring-bias. The material from which either the support frame 102 and/or the valve anchor 104 is fabricated can allow the support frame 102 and/or the valve anchor 104 to automatically expand to its functional size and shape when deployed but also allows the support frame 102 and/or the valve anchor 104 to be radially compressed to a smaller profile for delivery through the patient's vasculature. Examples of suitable materials for self-expanding components described herein (e.g., support frames, valve anchors, locking members) include, but are not limited to, medical grade nickel titanium alloys, tantalum, platinum alloys, niobium alloys, cobalt alloys, alginate, or combinations thereof. Shape memory alloys having superelastic properties generally made from ratios of nickel and titanium, commonly known as nitinol, are preferred materials. In some embodiments, self-expanding components described herein can include materials including, but not limited to shape memory plastics, polymers, and thermoplastic materials which are inert in the body. In an alternative embodiment, either the support frame 102 and/or the valve anchor 104 is not self-expanding, and may be expanded, for example, using a balloon catheter as is well known in the art. Examples of suitable materials for components described herein include, but are not limited to, stainless steel and titanium. Optionally, either the support frame 102 and/or the valve anchor 104 can comprise radiopaque materials to allow visualization under fluoroscopy or other imaging techniques.

Optionally, the support frame 102 can comprise one or more hooks 109 that can engage with tissue of the native valve annulus, the aortic root, or any other portion of the native valve when the support frame 102 is expanded within the native valve annulus. The hooks 109 can be engaged with the native valve annulus to secure the prosthesis 100 to mitigate any downstream or antegrade migration of the prosthesis 100 during operation.

The support frame 102 can comprise a first end portion 110 and a second end portion 112. The first end portion 110 can be positioned upstream of the second end portion 112 when the prosthesis 100 is released within the native valve annulus. As illustrated in FIG. 2, the first end portion 110 of the support frame 102 can be shaped as a generally flat end of a cylinder, where first apices 114 of the support frame 102 lie generally in a common plane, which can be oriented substantially perpendicular relative to a longitudinal axis 120 of the prosthesis 100. Further, the second end portion 112 can be shaped to include a series of peaks 130 and valleys 132, where second apices or minor peaks 136 of the support frame 102 collectively form contours of the peaks 130 and valleys 132. The peaks 130 and valleys 132 of the second end portion 112 can be positioned downstream of the first end portion 110 when the prosthesis is seated within the native valve annulus.

In accordance with some embodiments, the prosthetic leaflets 106 can be coupled relative to the support frame 102 at locations circumferentially aligned with the peaks 130 of the second end portion 112, as shown in FIG. 2. In some embodiments, the prosthetic leaflets 106 can be coupled to the membrane 108, for example, using ultra-high molecular weight polyethylene sutures. This unique configuration can advantageously enable the prosthesis 100 to more fully approximate the native valve structures, permit a more natural blood flow without limiting or otherwise constraining movement of the valve leaflets 106, and more seamlessly integrate with surrounding architecture of the heart. In some embodiments, the prosthetic leaflets 106 can comprise features, including, but not limited to, planar features, flat features, three-dimensional features, Bézier curves, or other suitable shapes. Optionally, the prosthetic leaflets 106 can be shaped through fixation on a leaflet-shaped mandrel.

The valve anchor 104 can comprise at least one U-shaped member, sinus locator, valve positioner, or valve hanger 140 that extends about a longitudinal axis of the valve anchor 104. As illustrated in FIG. 2, the valve anchor 104 can comprise a plurality of lobes or U-shaped members 140, such as three U-shaped members 140, but can have fewer or more. In some embodiments, U-shaped members 140 can be configured to engage with or fit inside the posterior aortic sinus, the left aortic sinus, and the right aortic sinus of a native aortic valve. The U-shaped members 140 can each have a peak portion 142 and a base portion 144. The U-shaped members 140 can each comprise first and second legs 146, 148. The first and second legs 146, 148 of the adjacent U-shaped members 140 can be interconnected at the peak portions 142 thereof. Further, the U-shaped members 140 can comprise shapes other than a U-shape, such as a wave-shape, V-shape, W-shape, or zig-zag. Optionally, multiple valve anchors 104 can each comprise one or more U-shaped members 140, wherein the multiple valve anchors 104 cooperatively engage with the aortic sinus to anchor the valve prosthesis as described herein.

The valve prosthesis 100 can include a link mechanism 160 that interconnects the support frame 102 to the valve anchor 104. The link mechanism 160 can comprise a single, continuous strand of material or multiple, independent strands of material that interconnects the support frame 102 to the valve anchor 104. Further, the link mechanism 160 can attach in a sliding, engaged, or fixed manner to one or more locations on the support frame 102 and/or on the valve anchor 104.

In accordance with some embodiments, the valve anchor 104 may optionally define one or more engagement areas in one or more portions of the valve anchor 104, where a link mechanism 160 may engage with the one or more engagement areas to restrict relative motion between the support frame 102 and the valve anchor 104.

For example, at the interconnection of the respective peak portions, the valve anchor 104 can define an engagement area 150. The engagement area 150 may also be referred to as a peak portion engagement arca.

As illustrated in FIG. 2, the support frame 102 can be flexibly coupled to the valve anchor 104 via one or more link mechanisms 160. The link mechanism 160 can be coupled to the support frame 102 and to the valve anchor 104, permitting relative movement between the support frame 102 and the valve anchor 104. However, the link mechanism 160 can be configured to limit relative movement between the support frame 102 and to the valve anchor 104. In some embodiments, the engagement area 150 of the valve anchor 104 can be used to further restrict relative motion of the support frame 102 with respect to the valve anchor 104 when the link mechanism 160 is engaged in the engagement area 150, as discussed herein.

The valve anchor 104 can thus be coupled to the support frame 102 to permit the valve anchor 104 to be moved axially or longitudinally relative to the support frame 102 while still remaining coupled to the support frame 102. This advantageous feature of some embodiments can allow a clinician to independently position the valve anchor 104 relative to the support frame 102. For example, in a transcatheter aortic valve replacement, the clinician can independently position the valve anchor 104 in order to fit the base portions 144 of the valve anchor 104 into the aortic sinus. Portions of the of aortic sinus may include the posterior aortic sinus, the left aortic sinus, and/or the right aortic sinus, of a native aortic valve. In some embodiments, the valve anchor 104 can rotate to be aligned in the respective aortic sinuses. In some embodiments, the interconnection of the valve anchor 104 to the support frame 102 can allow the valve anchor 104 to be rotated or repositioned, such as to self-rotate, to be aligned in or relative to the aortic sinus. The longitudinal and rotational movement of the valve anchor 104 can be facilitated via the interconnection of engagement areas 150 of the valve anchor 104 with a controlled member or grasper of the delivery device, as discussed further below.

With the valve anchor 104 “landed” in the respective aortic sinuses, the interconnection of the valve anchor 104 to the support frame 102 further permits the support frame 102 to be translated along the longitudinal axis 120 of the valve prosthesis 100. In some embodiments, during the delivery procedure, the valve anchor 104 can be moved at least axially from a proximal position relative to the support frame 102, to a distal position relative to the support frame 102, or from either of such positions to a position in which the support frame 102 at least partially longitudinally overlaps with or is concentric within the valve anchor 104. A range of various positions are illustrated, for example, in U.S. Pat. Nos. 11,090,156 and 11,083,577, the entireties of each of which is incorporated herein by reference.

For example, when the support frame 102 is nested within the valve anchor 104, as shown in FIG. 2, the base portions 144 of the valve anchor 104 can be longitudinally spaced apart from first end portion 110 of the support frame 102 along the longitudinal axis 120 at a distance which is about 10% to about 100%, about 25% to about 75%, about 33% to about 100%, about 33% to about 66%, about 25% to about 75%, about 50% to about 75%, or about 60% to about 70% of a length of the support frame 102. In some embodiments, the support frame 102 can be contained or otherwise fully overlapping the valve anchor 104. In some embodiments, the support frame 102 can have minimal or no overlap with the valve anchor 104. The support frame 102 can move along the longitudinal axis 120 to overlap the valve anchor 104 by about 10% to about 100%, about 25% to about 75%, about 33% to about 100%, about 33% to about 66%, about 25% to about 75%, or about 50% to about 75% of the length of the support frame 102. In accordance with some embodiments, the U-shaped members 140 of the valve anchor 104 can be in nested positions within the aortic sinuses, and the base portions 144 of the valve anchor 104 can be about longitudinally adjacent to, coplanar with, or spaced apart from the first end portion 110 of the support frame 102. For example, the valve anchor 104 can be in a nested position when at least one base portion 144 of the valve anchor 104 is in contact with or adjacent to the basal attachments of the native aortic valvar leaflets. Further, the first end portion 110 of the support frame 102 can be longitudinally adjacent to, coplanar with, or spaced apart from the native valve structure (or a virtual ring formed by the basal attachments of the native aortic valvar leaflets) or with the ventriculo-aortic junction.

The link mechanism 160 can allow rotational and longitudinal movement of the valve anchor 104 relative to the support frame 102. Thus, despite the presence of the link mechanism 160, the valve anchor 104 can move rotationally with respect to the support frame 102. Further, in some embodiments, the link mechanism 160 can be fixedly attached or coupled to the support frame 102 and fixedly or slidably attached to the valve anchor 104. When the support frame 102 is moved relative to the valve anchor 104, the link mechanism 160 can slide along the U-shaped members 140. In some embodiments, the U-shaped members 140 have a generally arcuate or convex shape (as illustrated with the U-shaped members of FIG. 2) that allows unrestricted movement of the link mechanism 160 along the geometry of the first and second legs 146, 148 of the U-shaped members 140. When the link mechanism 160 is allowed to slide along the first and second legs 146, 148 of the U-shaped members 140, the valve prosthesis 100 can be in a position referred to as a “slidable” state. In the slidable state, the range of longitudinal and/or rotational movement of the support frame 102 relative to the valve anchor 104 is variable and may be its greatest because the link mechanism 160 can move along the first and second legs 146, 148 of the U-shaped members 140.

In some embodiments, the link mechanism 160 can be fixedly attached or coupled to the support frame 102 and fixedly attached to the valve anchor 104. When the support frame 102 is moved relative to the valve anchor 104, the link mechanism 160 can stretch, flex, deform elastically and/or plastically. As the link mechanism 160 deforms, the range of longitudinal and/or rotational movement of the support frame 102 relative to the valve anchor 104 is variable as allowed by the deformation of the link mechanism 160.

Referring now to FIG. 3, a side cross-sectional view is provided of the valve prosthesis 100 loaded onto the delivery device 200, according to some embodiments. Among the many features illustrated in FIG. 3, the delivery device 200 can comprise a distal carrier assembly 206 that at least partially houses the support frame 102. Further, FIG. 3 shows that a proximal enclosure 210 of delivery device 200 can extend over both the valve anchor 104 and the support frame 102. A distal enclosure 212 of the distal carrier assembly 206 can at least partially house at least the support frame 102. The proximal enclosure 210 can be coupled to a first core member 220, and the distal enclosure 212 can be coupled to a second core member 222. In some embodiments, the distal enclosure 212 can be threadedly and/or adhesively coupled or bonded to the second core member 222.

Thus, in accordance with some embodiments, in the compressed or delivery configuration shown in FIG. 3, the link mechanism (not shown) can extend between the valve anchor 104 and the support frame 102 and be at least partially enclosed within the proximal enclosure 210 (depending on the attachment point of the link mechanism with the support frame 102 and the longitudinal extent of the proximal enclosure 210). Other details of delivery devices and prostheses are provided in U.S. Pat. Nos. 11,090,156 and 11,083,577, the entireties of each of which is incorporated herein by reference.

In addition, FIG. 3 illustrates that the delivery system can be used to engage the engagement areas 150 of the valve anchor 104, for example, using a control member or grasper 224 to facilitate movement and control of the positioning of the valve anchor 104 during delivery. As discussed in U.S. Pat. Nos. 11,090,156 and 11,083,577, the entireties of each of which is incorporated herein by reference, this engagement can maintain the engagement areas 150 in a common plane, oriented generally perpendicular relative to the longitudinal axis of the delivery device 200.

In some embodiments, the delivery device can comprise a grasper mechanism. The grasper mechanism can have one, two, three, four, or more grasper arms or other such components that serve as a point of contact with a respective point of contact of the valve prosthesis, such as the valve frame or valve anchor of the prosthesis. Thus, the grasper mechanism can be used to securely couple a portion of the valve anchor with the delivery device to permit the clinician to control movement, operation, and deployment of the valve anchor. The grasper mechanism can engage one or more portions or structures of the valve anchor using a variety of coupling mechanisms, which can use attachment means including mechanical engagement, dissolvable structures, chemically reactive degradable structures, electrolytically degradable structures, and the like.

In some embodiments, by engaging the valve anchor, e.g., at a base portion thereof, the grasper(s) can precisely engage with and control the longitudinal position of the valve anchor. Thus, the grasper(s) can be used to control the articulation of the valve anchor as desired by the clinician. For example, the grasper(s) can each have independent translational actuation (e.g., relative to each other) that can collectively “steer” the valve anchor relative to and away from or towards a central or delivery axis of the system. Additionally or optionally, collective and/or individual actuation of the grasper(s) can be used to indirectly or directly control or affect the position, shape and/or movement of the valve frame.

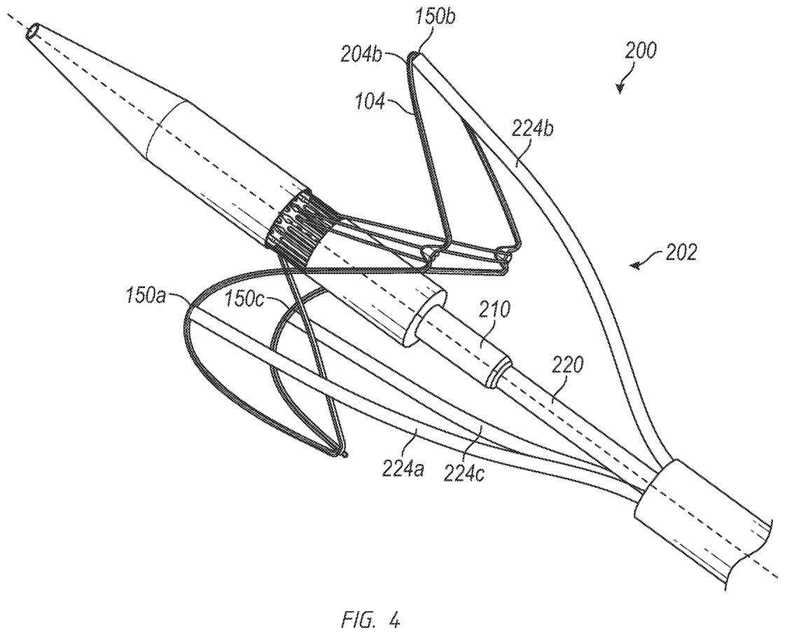

FIG. 4 illustrates aspects of the delivery device 200, according to some embodiments. These figures do not illustrate all of the components of the delivery device that can be incorporated into an embodiment. However, the features illustrated in these figures can be incorporated into embodiments of the delivery device to facilitate engagement with the valve anchor and/or facilitate delivery and control of the valve anchor during implantation and release of the valve anchor at the target location.

For example, FIG. 4 illustrates an embodiment of a delivery device 200 that comprises a grasper mechanism 202. The delivery device 200 is shown with the valve anchor 104 in a partially opened, yet still constrained (“partially deployed”) configuration. In some embodiments, the grasper mechanism 202 can comprise at least one grasper arm. In some embodiments, the grasper arm can comprise a tubular enclosure or structure. As shown, the delivery device 200 can have three grasper arms 224a, 224b, 224c that can engage with and control the longitudinal position of the valve anchor 104.

In some embodiments, each grasper arm 224a, 224b, 224c of the delivery device 200 can comprise an engagement wire that is movable within a lumen of a tubular enclosure. The valve anchor 104 can be configured to comprise an anchor tab extending from an engagement area 150a, 150b, 150c of the valve anchor 104.

In some embodiments, the engagement wire can interconnect with one or more portions of the valve prosthesis to provide a releasable connection therewith.

For example, the engagement wire can be releasably coupled to a hole, protrusion, or other structure of the valve anchor. The engagement wire can loop through or around an engagement area, such as an anchor tab, of the valve anchor and be released upon actuation of the clinician to permit separation of the valve prosthesis from the delivery device.

In some embodiments, the engagement wire can comprise a distal end portion that includes pins, ridges, or protrusions that can be coupled to the engagement structure of the anchor tab at the engagement area of the valve anchor. When engaged together, the engagement wire and the anchor tab can be proximally drawn into the lumen of the tubular enclosure, which secures the engagement wire and the anchor tab relative to each other in both radial and longitudinal directions. However, when the engagement wire and the anchor tab are moved outside of the lumen of the tubular enclosure, the engagement wire and the anchor tab can be disengaged as the valve anchor and the anchor tab expand radially, thereby disengaging the anchor tab from the engagement wire. Some aspects of the delivery systems, prosthetic valves, and delivery methods disclosed herein can be implemented in accordance with features disclosed in Applicant's U.S. Pat. Nos. 11,090,156 and 11,083,577, the entireties of each of which is incorporated herein by reference.

The wire can be constructed of a metal or non-metal material. In some embodiments, the valve anchor can comprise a surface treated (passivated) nitinol. In such embodiments, it may be advantageous to have the wire be of non-metal material so as not to disrupt the surface. As discussed herein, the arrangement and position of the wire can allow the wire to maintain engagement with the valve anchor rather than requiring or relying on a high-friction engagement between the wire and the valve anchor. Thus, in some embodiments, the wire can comprise a non-metallic material with a low coefficient of friction against the valve anchor, thus permitting the wire to move smoothly and freely upon intentional actuation by the clinician while avoiding unintentional movement thereof.

Alternatively, the wire can comprise a metal, such as nitinol, which can be advantageous because nitinol is less prone to kink and plastic deformation. However, steel or other materials can also be used.

In some embodiments, the wire can have a diameter of about 0.020″ for most of the length to provide a stiffness, reduced stretching, and provide responsiveness to proximal actuation during release.

In some embodiments, the wire can be constructed of a centerless ground nitinol wire at the distal end portion (e.g., a 30-50 mm length of wire ground at the distal tip from a 0.020″ diameter wire to 0.008″ diameter; the grind can happen over a conical transition). In some embodiments, the ground wire can be what connects to the anchor tab.

The wire can be constructed as: a centerless ground wire (continuous piece of material); a welded wire (e.g., connecting two different diameter wires via a weld, such as a laser weld); a hypotube connected with a small wire on the center and another wire that is soldered, brazed, or bonded near a distal end portion of the hypotube (which can provide the advantage of stiffness of the hypotube and the flexibility of the distal portion using a wire); a wire that is doubled up or folded over on itself with the two ends being offset (one of the end portions extends distally to permit engagement with the valve anchor).

In accordance with some embodiments, engagement between the delivery device and the valve prosthesis, such as by using the anchor tab, can allow for a control member or grasper to engage with the valve anchor for a transfemoral retrograde delivery approach or for an antegrade delivery approach (as shown in U.S. Pat. No. 11,090,156 or U.S. Pat. No. 11,083,577). For example, the anchor tab can receive a control member or a grasper approaching the valve anchor proximal to the peak portions to allow the valve anchor to be used for a retrograde (e.g., a transfemoral retrograde) approach in delivering the valve anchor. Further, the anchor retention component can receive the control member or the grasper approaching the valve anchor proximal to the first and second U-shaped members to allow the valve anchor to be used for an antegrade, apical, or transapical approach in delivering the valve anchor.

Various configurations of engagement areas of the anchor tab of the valve anchor can be used in some embodiments disclosed herein. Further, in any of the embodiments disclosed herein, the engagement area can comprise barbs or hooks over which a link mechanism can pass, as discussed in U.S. Pat. No. 11,083,577, the entirety of which is incorporated herein by reference. The barbs or hooks can permit one-way motion of the link mechanism-once the link mechanism crosses the barbs or hooks, the barbs or hooks will prevent reverse movement of the link mechanism over the barbs or hooks. The illustrated embodiments provide double peaks or coves that can tend to capture the link mechanism during the stages of prosthesis delivery, as discussed herein. When the link mechanism is so captured, the valve prosthesis 100 can be in a position referred to as a “retained” position.

During use, after the valve anchor has been released from within the proximal sheath and after the valve anchor and the valve frame have been released from the delivery device, the grasper mechanism of the delivery device can be configured to be compactly reassembled and withdrawn into the introducer sheath in order to minimize any damage to the blood vessel through which the delivery device was advanced.

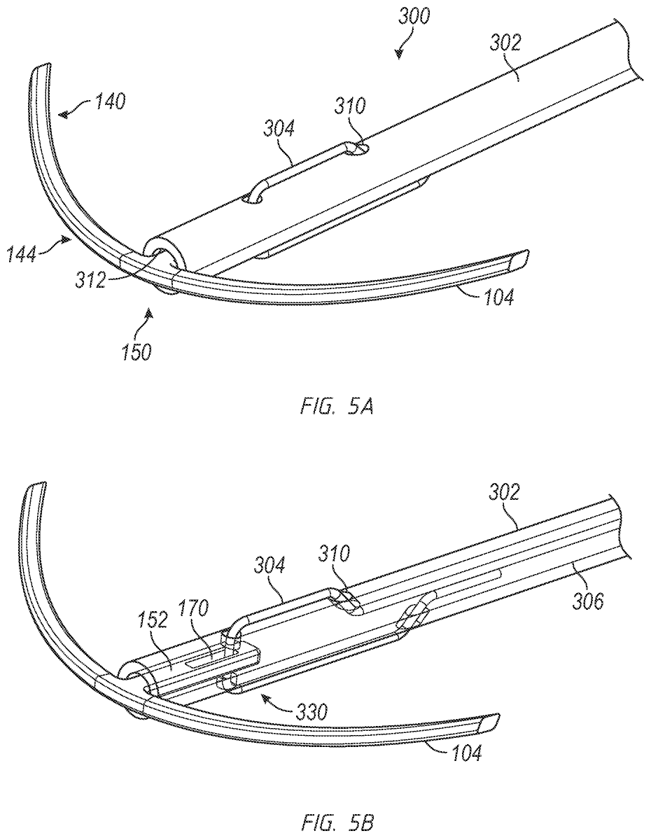

Referring now to FIGS. 5A-7D, a first embodiment of the engagement between a grasper mechanism 300 and a valve anchor 104 is shown in various views and in an engaged position and a disengaged position. FIGS. 5A and 5B illustrate that the grasper mechanism 300 can comprise a tubular member 302 (shown in FIG. 5B is a transparent component) and an engagement wire 304.

The engagement wire 304 can extend within a lumen 306 of the tubular member 302. The tubular member 302 can comprise one or more side apertures or holes 310 through which the engagement wire 304 can pass in order to form an interlocking engagement with the valve anchor 104. For example, in some embodiments, the engagement wire can extend within a proximal section of the tubular member and exit the lumen along a distal section thereof to facilitate engagement with a valve anchor.

As shown in the embodiment of FIGS. 5A-7D, the tubular member 302 can comprise a plurality, such as two, three, four, five, or more, apertures that permit the engagement wire 304 to engage with the valve anchor 104. For example, the engagement wire 304 can exit and reenter the lumen 306 to facilitate engagement with an anchor tab 152 of the valve anchor 104.

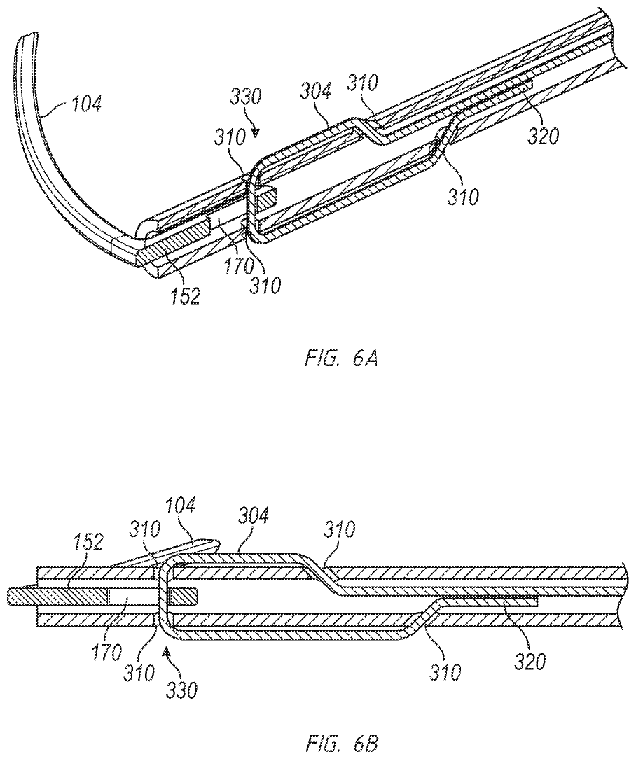

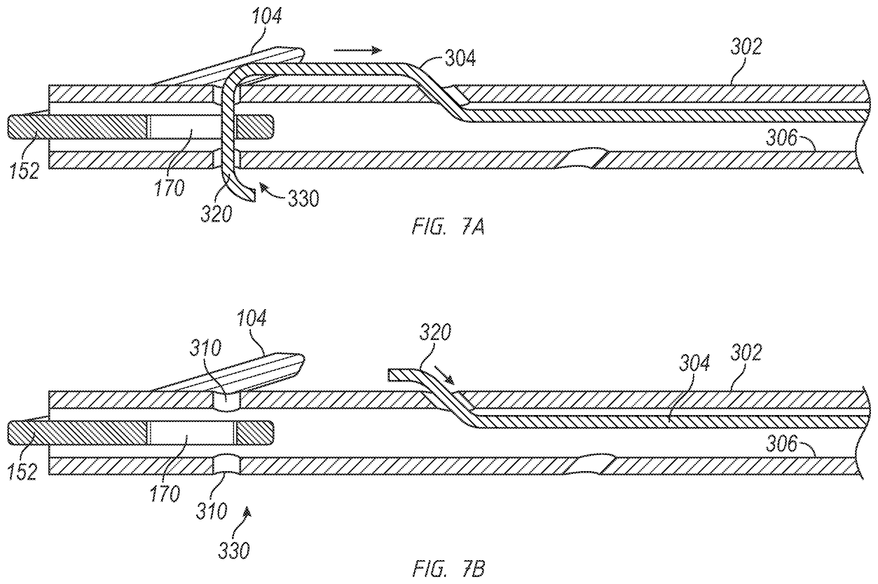

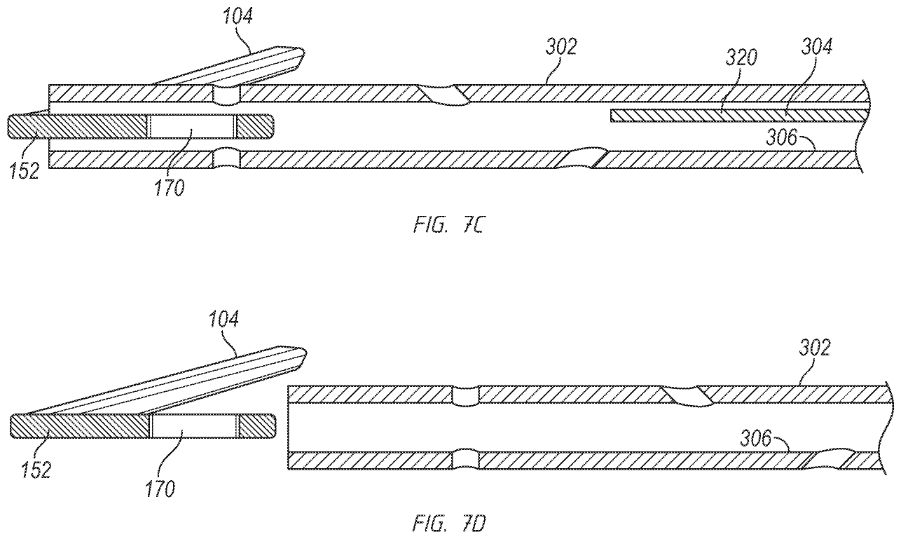

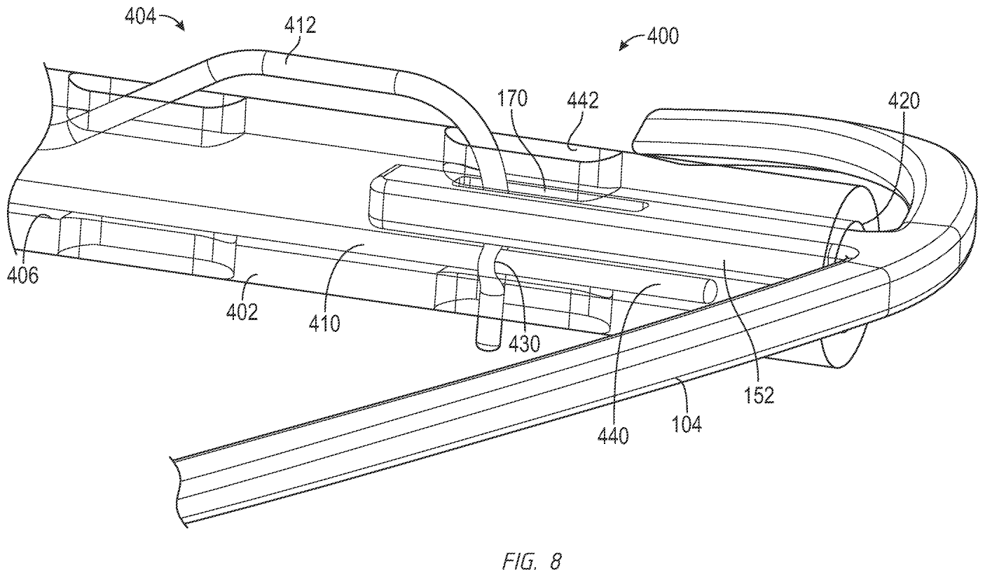

The anchor tab 152 can enter the lumen 306 of the tubular member 302 through an end opening 312 of the tubular member 302. When positioned therein, the anchor tab 152 can be engaged with the engagement wire 304. For example, as shown in FIGS. 5B-6B, the engagement wire 304 can pass through a slot or aperture 170 of the anchor tab 152. In this configuration, the engagement wire 304 can maintain the anchor tab 152 in an engaged position that ensures that the grasper mechanism 300 is engaged securely with the valve anchor 104. However, upon actuation by the clinician, the engagement wire 304 can be proximally withdrawn within the lumen 306 of the tubular member 302. As the engagement wire 304 is proximally withdrawn, an end portion 320 of the engagement wire 304 will be pulled through the apertures 310 of the tubular member 302 until the end portion 320 of the engagement wire 304 is also pulled through the slot 170 of the anchor tab 152. This gradual release or disengagement process is illustrated in FIGS. 7A and 7B. Upon continued proximal withdrawal of the engagement wire 304 through the lumen 306, the engagement wire 304 can be pulled back into the lumen 306 of the tubular member 302 and the anchor tab 152 will be disengaged from the engagement wire 304.

The apertures 310 can comprise one or more proximally positioned apertures and/or one or more distally positioned apertures. Various embodiments are illustrated in FIGS. 5A-10 that utilize such apertures that can permit access to a lumen of the tubular member for facilitating engagement with the valve anchor.

For example, FIGS. 5A-7D illustrate an embodiment in which the apertures are arranged, as noted in FIG. 6B, with first and second distal apertures 310a, 310b and first and second proximal apertures 310c, 310d. In some embodiments, the first distal aperture 310a can be diametrically opposed relative to the second distal aperture 310b. In some embodiments, the first proximal aperture 310c can be diametrically opposed relative to the second proximal aperture 310d. However, the first proximal aperture 310c can be longitudinally offset from the second proximal aperture 310d.

In order to engage the valve anchor, the engagement wire can extend through one or both of the distalmost pair of apertures 310a, 310b and/or one or both of the distalmost pair of apertures 310a, 310b, and engage with the valve anchor in the engagement arca, as discussed herein.

In some embodiments, the end portion 320 of the engagement wire 304 can be tucked into the first proximal aperture 310c, as shown in FIG. 6B. As such, this arrangement may be useful to help avoid having the end portion 320 inadvertently extend into another of the apertures 310a, 310b, or 310d (or others).

However, in some embodiments, it may be advantageous to have the final aperture (i.e., the aperture through which the end portion 320 of the engagement wire 304 passes when entering the lumen 306) be the second proximal aperture 310d. Thus, the arrangement in FIG. 6B can be reversed such that the wire 304 initially exits the lumen 306 through the first proximal aperture 310c, then reenters the lumen 306 through the second distal aperture 310b to engage with the slot 170, then exits the lumen 306 via the first distal aperture 310a, and finally reenters the lumen 306 via the second proximal aperture 310d. In such an embodiment, the positioning of the wire 304 through these apertures can thus allow for shorter or quicker positional timing release of the end portion 320 of the wire 304 from the second proximal aperture 310d.

In accordance with some embodiments, the aperture 310 can each have a longitudinal axis that extends substantially perpendicular relative to a longitudinal axis of the tubular member. For example, as shown in FIG. 6B, the first and second distal apertures 310a, 310b can each have a longitudinal axis that extends substantially perpendicular relative to a longitudinal axis of the tubular member.

Alternatively, however, in some embodiments, as shown by the first and second proximal apertures 310c, 310d of FIG. 6B, the apertures 310 can have a longitudinal axis that extends transversely relative to a longitudinal axis of the tubular member. As shown in FIG. 6B, the first and second proximal apertures 310c, 310d can each have a longitudinal axis that extends transverse relative to a longitudinal axis of the tubular member. This transverse alignment of the longitudinal axis can facilitate movement of the wire 304 through the aperture.

According to some embodiments, the multi-aperture arrangement can enable the wire 304 to be oriented transverse or perpendicular relative to the direction of displacement of the tab when being pulled out of the lumen 306. The embodiments shown can permit the wire to remain statically positioned and retain or engage the tab, even if the end portion of the wire is inadvertently dislodged from the final aperture.

Thereafter, as shown in FIGS. 7C and 7D, the valve anchor can begin to be separated from the grasper mechanism 300 such that the anchor tab 152 is fully withdrawn from the lumen 306 of the tubular member 302. Once the anchor tab 152 has exited the lumen 306 of the tubular member 302, the valve anchor 104 can freely radially expand relative to the delivery device 200 (not shown).

Among the many advantages associated with the embodiment illustrated in FIGS. 5A-7D is the secure engagement between the grasper mechanism and the valve anchor 104 while also permitting case of disengagement. The secure engagement can be achieved in accordance with the novel embodiments disclosed herein given the increased ability to have precise tolerances between a fitted length of the anchor tab within the lumen (e.g., from the engagement area of the valve anchor to a distal end edge of the anchor tab slot) and a length 324 of the apertures of the tubular member from the end opening thereof, which can be matched to provide precise alignment of the slot with the apertures and minimize chatter when the anchor tab is inserted in the lumen and engaged by the engagement wire.

For example, in some embodiments, a distalmost set of apertures 330, as illustrated in FIGS. 6A-7B, can be positioned at a substantially equal longitudinal position along and axis of the tubular member 302 such that proximal most edges of the slot 170 and the distalmost set of apertures 330 are positioned adjacent to each other or at a substantially common longitudinal position along the axis of the tubular member 302. This relative positioning of the proximalmost edges of the slot 170 and the distalmost set of apertures 30 can ensure that when the anchor tab 152 is fully inserted into the end opening 312, the positions of these edges can be aligned and engage against the engagement wire 304 to limit distal migration of the anchor tab 152 out of the lumen 306 of the tubular member 302. Accordingly, through the alignment and positioning of the distalmost set of apertures 330 and the slot 170 of the anchor tab 152, the base portion 144 of the valve anchor 104 can firmly abut and be retained against the distal end of the tubular member 302 until the engagement wire 304 is proximally withdrawn into the lumen 306, thereby releasing the valve anchor 104 from engagement with the grasper mechanism 300.

These and other features disclosed herein provide an innovative design that enables a clinician to enjoy the benefits of both secure engagement of the valve anchor until the release of the valve anchor is needed and certainty that the valve anchor will be released without causing damage to the valve anchor or native valve structure (e.g., by requiring a torsional, tensile, or bending force or other damaging movement) that may in advertently occur during release of the valve anchor.

Moreover, in accordance with at least some embodiments disclosed herein is the realization that these challenges have been unresolved despite years of research and development by numerous companies at great cost. The present Applicant, by the embodiments disclosed and suggested herein, has been able to successfully achieve a solution that successfully minimizes or prevents inadvertent release of the valve anchor or heart prosthesis during delivery and ensures that the heart valve prosthesis can be released with precision at the target location to avoid any trauma or misalignment of the valve prosthesis during delivery.

Referring now to FIG. 8, another embodiment of a grasper mechanism is illustrated. Similar to the embodiment shown in FIGS. 5A-7D, the embodiment illustrated in FIG. 8 can also comprise a tubular member having a plurality of apertures and an engagement wire that can pass through the apertures and engage with a portion of a valve anchor.

As illustrated in FIG. 8, a grasper mechanism 400 can comprise a tubular member 402 (shown as a transparent component) and an engagement wire mechanism 404 that can extend at least partially within a lumen 406 of the tubular member 402. While the grasper mechanism 400 can share many of the same principles of operation and features as the grasper mechanism 300 discussed above, the engagement wire mechanism 404 provides a simple alternative structure and function. Accordingly, the discussion of features and advantages of the grasper mechanism 300 can also apply to the grasper mechanism 400 will not be repeated herein for brevity.

With regard to the engagement wire mechanism 404, some embodiments can provide multiple components, such as a two-part structure that allows for independent actuation of components in order to provide additional surety and discrete steps in the release process.

For example, the engagement wire mechanism 404 can comprise a pull wire 410 and a loop wire 412. The pull wire 410 and the loop wire 412 can intersect and engage with each other at a distal location (e.g., adjacent to the docking area were along the valve anchor is engaged with the grasper mechanism) along the grasper mechanism 400. As illustrated, the pull wire 410 and the loop wire 412 can intersect and create a closed loop or by the engagement wire mechanism 404 engages with a slot 170 of the valve anchor 104.

Accordingly, similar to the embodiment discussed above respect to the grasper mechanism 300, an anchor tab 152 of the valve anchor 104 can be inserted into an end opening 420 of the tubular member 402 and the pull wire 410 and/or the loop wire 412 can pass through the slot 170 of the anchor tab 152 for restricting axial movement of the anchor tab 152 out of the opening 420 until the engagement wire mechanism 404 is disengaged by the clinician, thereby allowing the grasper mechanism 400 to disengage from the valve anchor 104.

In accordance with some embodiments, the loop wire 412 can comprise an engagement structure, such as an aperture structure 430 that extends through or is coupled to the loop wire 412. As illustrated, the loop wire 412 can be configured to incorporate or be coupled to the aperture structure 430 along a distal end portion of the loop wire 412. The aperture structure 430 can be configured to receive the pull wire 410 therethrough, thereby forming a loop that can engage with the slot 170 of the valve anchor 104.

In order to disengage the engagement wire mechanism 404 from the slot 170, the clinician can proximately retract the pull wire 410, which will cause the pull wire 410 to eventually separate and withdraw from the aperture structure 430 to separate the pull wire 410 from the loop wire 412. Once separated, the loop wire 412 can be proximately retracted within the lumen 406 and exit from the slot 170. In operation, both the pull wire 410 and the loop wire 412 can be proximately retracted and be positioned entirely within the lumen 406 after disengagement from the valve anchor 104. Further, the anchor tab 152 can exit the end opening 420 in order to permit the valve anchor 104 to freely expand at the target location.

In accordance with at least some embodiments disclosed herein is the realization that the grasper mechanism 400 can permit the clinician to operate separate controls or actuators that control movement of the pull wire 410 and the loop wire 412, thereby establishing discrete steps of disengaging the pull wire 410 from the loop wire 412 and thereafter permitting the clinician to conduct a final disengagement of the loop wire 412 from the slot 170. These actions can be performed via rotation or translation of an actuator or knob of the control handle of the delivery device. This discrete step of disengaging the pull wire 410 from the loop wire 412 can offer additional certainty and control to the clinician, thereby minimizing any inadvertent disengagement or minimizing ambiguity as to when or whether the disengagement process has begun. This can be advantageous during the placement procedure so that a clinician is given another discrete step in the disengagement process.

Additionally, some embodiments of the grasper mechanism 400 can be implemented such that the pull wire 410 comprises a distal end portion 440 that extends beyond the aperture structure 430 to a position adjacent to the end opening 420, or within about 1 mm, about 2 mm, about 3 mm, about 4 mm, or about 5 mm from the end opening 420. The dimensions and configuration of the distal end portion 440 can depend on the axial length of the slot 170, the position of the site aperture 442 of the tubular member 402, and the gauge of the pull wire 410. Indeed, in accordance with some embodiments, the pull wire 410 will provide sufficient rigidity and be able to withstand any lateral bending force exerted thereon by the loop wire 412 so that the pull wire 410 does not get pulled through the slot 170 and disengage from the loop wire 412 inadvertently.

Further, in accordance with some embodiments, the aperture structure 430 can have an inner profile or dimension that closely matches an outer profile or dimension of the pull wire 410. In accordance with some embodiments, the outer cross-section or profile of the loop wire 412 and the aperture structure 430 can be less than a corresponding dimension or profile of the slot 170 in order to permit the loop wire 412 to be pulled through the slot 170.

For example, in accordance with some embodiments, the aperture structure 430 can have an expanded configuration when the pull wire 410 is inserted therethrough (which expanded configuration can be larger than the corresponding dimension or profile of the slot 170), thereby preventing the aperture structure 430 from passing through the slot 170 in the expanded configuration. However, after the pull wire 410 is removed from the aperture structure 430, the aperture structure 430 can be compressed or automatically returned to a collapsed configuration that is capable of passing through the slot 170. Accordingly, the configuration of the aperture structure 430 can facilitate engagement or disengagement of the engagement wire mechanism 404 with the anchor tab 152.

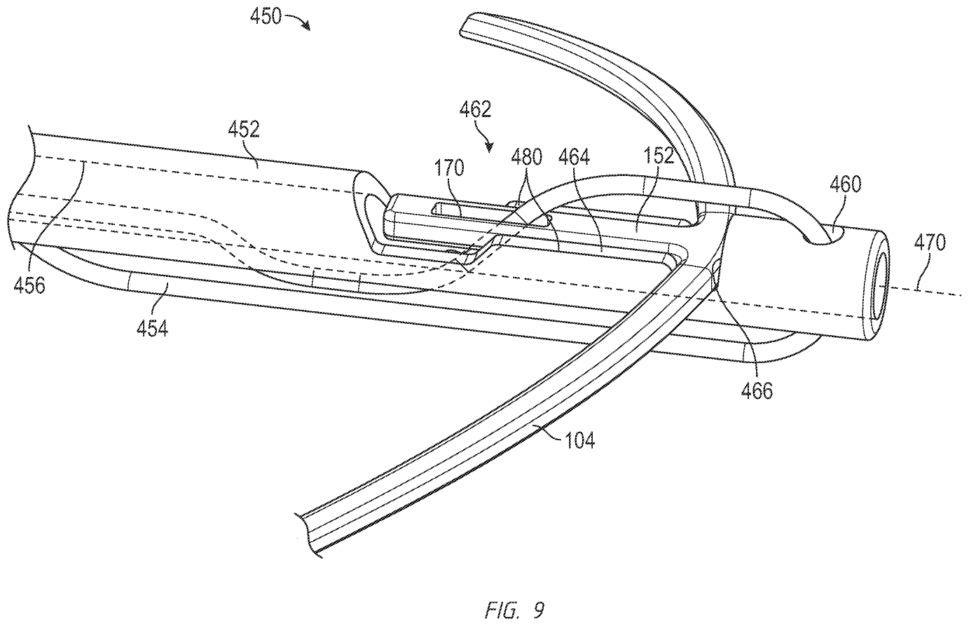

Referring now to FIG. 9, an additional embodiment of a grasper mechanism 450 is illustrated. The grasper mechanism 450 can comprise a tubular member 452 and an engagement wire 454. Similar to the embodiment discussed above with respect to FIGS. 5A-7D, the engagement wire 454 can comprise a single continuous wire that passes through a lumen 456 of the tubular member 452 and passes through apertures 460 of the tubular member in order to form a loop that engages with a portion of a valve anchor 104. In accordance with some embodiments, the grasper mechanism 450 can be configured such that the tubular member 452 comprises a slot or cut out 462 along an outer surface of the tubular member 452. The slot or cut out 462 can comprise or form a receptacle 464 having one or more structures that are formed to be complementary to a shape or structure of valve anchor 104 and/or the anchor tab 152.

For example, the receptacle 464 shown in FIG. 9 can comprise a shoulder area 466 configured to receive the valve anchor 104 therein and restrict longitudinal or axial movement of the valve anchor 104 relative to the tubular member 452. For example, the shoulder area 466 can comprise a depression or well that allows the valve anchor 104 to be positioned therein and that restricts degrees of freedom of movement of the valve anchor 104 except for a translational, radial degree of motion that is generally perpendicular relative to a longitudinal axis 470 of the tubular member 452. Accordingly, unless otherwise constrained, the valve anchor 104 can exit the shoulder area or receptacle 464 in a direction radially away from the longitudinal axis 470, but would be otherwise constrained against motion in other directions (translational or rotational).

In some embodiments, the engagement wire 454 can constrain the radial motion of the valve anchor 104 and thereby provide security engagement between the valve anchor 104 and the grasper mechanism 450. For example, the engagement wire 454 can loop around the anchor tab 152 and into the slot 170 of the anchor tab 152 thereby engaging the anchor tab 152 and the valve anchor 104 to restrict radial movement of the valve anchor in a direction away from the longitudinal axis 470 and out of engagement with the shoulder arca 466.

Optionally, the tubular member 452 can be configured such that the receptacle 464 comprises opposing rail portions 472 that extend longitudinally away from the shoulder arca 466 and define a gap therebetween that is configured to receive at least a portion of the valve anchor (e.g., the anchor tab 152) therein. The rail portions 472 can thereby aid in restricting degrees of motion of the anchor tab 152 and the valve anchor 104 relative to the tubular member 452.

Among the advantages associated with the embodiment of the grasper mechanism 450 illustrated in FIG. 9 is the increased facility of disengagement and separation of the valve anchor 104 from the tubular member 452. For example, the delivery device can be configured such that the receptacles 464 of the grasper mechanism 450 (especially when multiple grasper mechanisms 450 are used, such as in the embodiment of the delivery device shown in FIG. 4, i.e., when three grasper mechanisms are used), the receptacle 464 can face radially outwardly relative to a longitudinal axis of the delivery device. Accordingly, when the receptacle 464 faces radially outwardly or is open in a direction away from the longitudinal axis of the delivery device, the valve anchor 104 can expand in a direction radially away from the grasper mechanism 450 with case. In this manner, once the engagement wire 454 is proximally withdrawn by the clinician and disengaged from the slot 170, the valve anchor 104 can immediately release from the tubular member 452 and expand into apposition with the native tissue of the target area. Thus, the grasper mechanism 450 will not tend to interfere with expansion of the valve anchor 104.

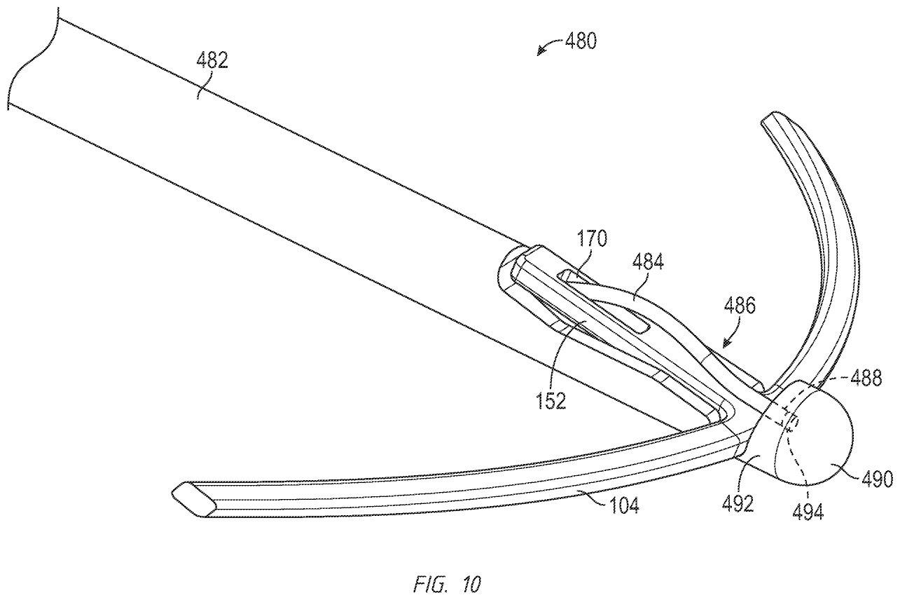

Referring now to the embodiment illustrated in FIG. 10, similar to the embodiment illustrated in FIG. 9, a grasper mechanism 480 can comprise a tubular member 482 and an engagement wire 484 that can collectively engage a valve anchor 104. Similar to the grasper mechanism 450, the grasper mechanism 480 can comprise a receptacle 486 configured to receive at least a portion of the valve anchor 104, such as the anchor tab 152, and to restrain degrees of motion of the valve anchor 104 relative to the tubular member 482. Various features and functions of the tubular member 482 and the receptacle 486 are similar to those discussed above with regard to the tubular member 452 and the receptacle 464 shown in FIG. 9 and will not be repeated herein for brevity, but are incorporated by reference.

In contrast to the embodiment of the grasper mechanism 450 shown in FIG. 9, the grasper mechanism 480 can be configured such that the engagement wire 484 comprises a distal end segment 488 that is removably coupled to an end cap 490 disposed at a distal end of the tubular member 482. The distal end segment 488 of the engagement wire 484 can be attached to the end cap 490. This attachment can be via an adhesive or mechanical means. In some embodiments, the end cap 490 can be formed onto the distal end segment 488 of the engagement wire 484 prior to the assembly of the grasper mechanism 480 and the valve anchor 104. Further, the end cap 490 can be separate from and attached to a distal portion 492 of the tubular member 482 such that when the engagement wire 484 is pulled proximally and separated from the end cap 490, the end cap 490 remains coupled to the distal portion 492 of the tubular member 482.

In accordance with some embodiments, the end cap 490 and the distal portion 492 of the tubular member 482 can be separate components that are attached or coupled to each other during the assembly of the delivery device with the part valve prosthesis. However, the end cap 490 and the distal portion 492 can form a single feature of the tubular member 482 having or forming a cavity that is configured to receive the distal end segment 488 of the engagement wire 484. In some embodiments, the distal end segment 488 may be positioned within the cavity 494 with or without an adhesive securing the engagement wire 484 to the inner surface of the cavity 494. Accordingly, with sufficient rigidity, the engagement wire 484 can serve to restrict radial movement of the valve anchor 104 away from a longitudinal axis of the tubular member 482 (at least in part due to the sheer strength of the engagement wire 484 adjacent to its insertion into the cavity 494). Moreover, with a releasable adhesive to provide a degree of engagement, with no adhesive (thus only frictional engagement), or with an interference fit (and high frictional engagement), the distal end segment 488 of the engagement wire 484 can be fitted and/or positioned within the cavity 494 in a manner to mitigate inadvertent or unintentional separation of the distal end segment 488 from the cavity 494, which can be overcome by an intentional proximal force exerted on the engagement wire 484 during release of the valve anchor 104. Other advantageous features of the grasper mechanism 480 are similar to those discussed above with respect to the embodiment illustrated in FIG. 9 and will not be repeated herein for brevity.

Accordingly, in the embodiment of the grasper mechanism 480 illustrated in FIG. 10, the engagement wire can be proximally pulled and, upon exertion of sufficient proximal force, separated from its coupling with the end cap 490. The engagement wire 484 can then be proximally withdrawn into the tubular member 482 and thereby permit disengagement and separation of the valve anchor 104 from the tubular member 482.

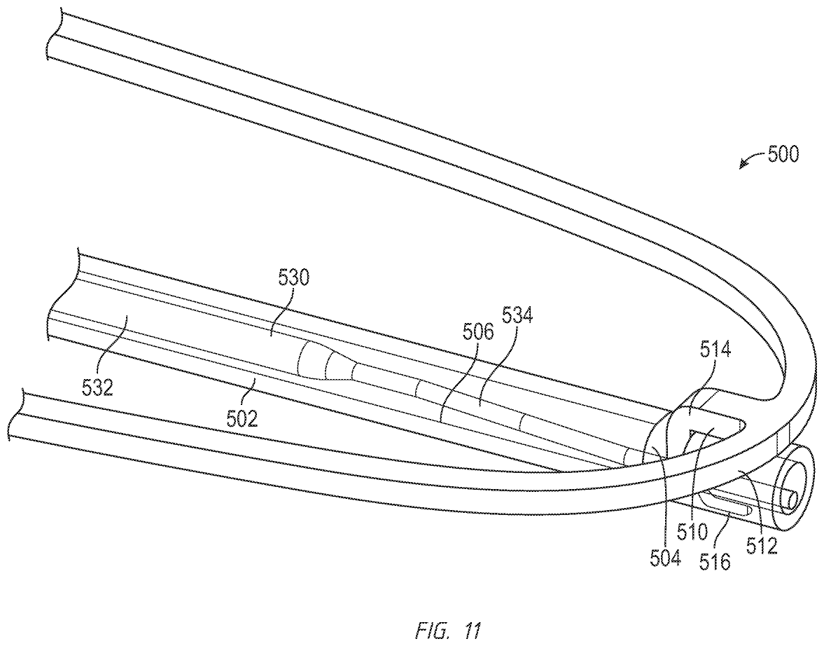

In accordance with yet another embodiment of the grasper mechanism disclosed herein, FIG. 11 illustrates a grasper mechanism 500. The grasper mechanism 500 can comprise a tubular member 502 (shown as a transparent component) having a side aperture 504 through which an anchor tab 510 of a valve anchor 512 can be inserted and engaged with an engagement wire 530. The grasper mechanism 500 can provide certain advantages over other embodiments disclosed herein, including simplicity of design, minimal components, and direct and simplified movements of the components thereof.

The engagement wire 530 can comprise a proximal section 532 and a distal section 534. The proximal section can define a dimension or gauge different from the distal section 534. In some embodiments, the proximal section 532 can have a smaller gauge than the distal section 534. As noted above, in some embodiments, the wire 530 can be ground, formed of different diameter wires and/or a hypotube that are coupled to each other, folded over on itself, or combinations thereof, to achieve a difference in the diameters of the proximal section 532 and the distal section 534. This difference in size or gauge of the proximal section 532 and the distal section 534 can allow the clinician to have a higher degree of responsiveness (less or no longitudinal stretching) when proximally retracting or exerting a proximal force on the engagement wire 530 due to the greater tensile strength of the proximal section 532 relative to the distal section 534. Nevertheless, the distal section 534 can also provide sufficient tensile strength in order to securely disengage from the anchor tab 510, as discussed herein.

In accordance with the embodiment disclosed in FIG. 11, the anchor tab 510 can be pre-bent to a generally right-angle configuration or bended into the side aperture 504 during assembly. Accordingly, the anchor tab 510 can be identical to the anchor tab 152 illustrated and described in various other embodiments disclosed herein except for the configuring of the anchor tab 510 in a direction generally transverse relative to a longitudinal axis of the anchor tab 510 or the longitudinal axis of the grasper mechanism 500.

As illustrated in FIG. 11, the bend of the anchor tab 510 can form a plug or insertion portion 514 of the anchor tab 510 that can be inserted through the side aperture 504 into a lumen 506 of the tubular member 502. The anchor tab 510 can comprise a slot 516 that can enter the lumen 506 at a depth sufficient to permit the distal section 534 of the engagement wire 530 to be inserted through the slot 516. In this manner, the engagement wire 530 can engage the anchor tab 510 and constrain movement of the anchor tab 510 such that the anchor tab 510 is not permitted to exit the side aperture 504.

In operation, the clinician can proximally withdraw the engagement wire 530 in order to disengage the distal section 534 of the engagement wire 530 from the slot 516 of the anchor tab 510. Once disengaged, the anchor tab 510 can move freely relative to the tubular member 502, thus permitting radial expansion of the valve anchor 512. As noted above with respect to the grasper mechanism 450 shown in FIG. 9, the side aperture 504 can be open in a direction away from a longitudinal axis of the delivery device such that when released, the valve anchor 512 can expand freely away from the grasper mechanism 500. Thus, the grasper mechanism 500 will not tend to interfere with expansion of the valve anchor 512.

In accordance with some embodiments, the grasper mechanism and delivery device disclosed herein can also be configured such that the anchor tab of the valve anchor includes a notch or protrusion that can longitudinally overlap with or fit against a corresponding protrusion or notch at a distal end section of an engagement wire. When longitudinally overlapping and constrained within the lumen of the tubular member, the anchor tab and the engagement wire can also be constrained against longitudinal movement relative to each other, thereby securing the anchor tab to the grasper mechanism.

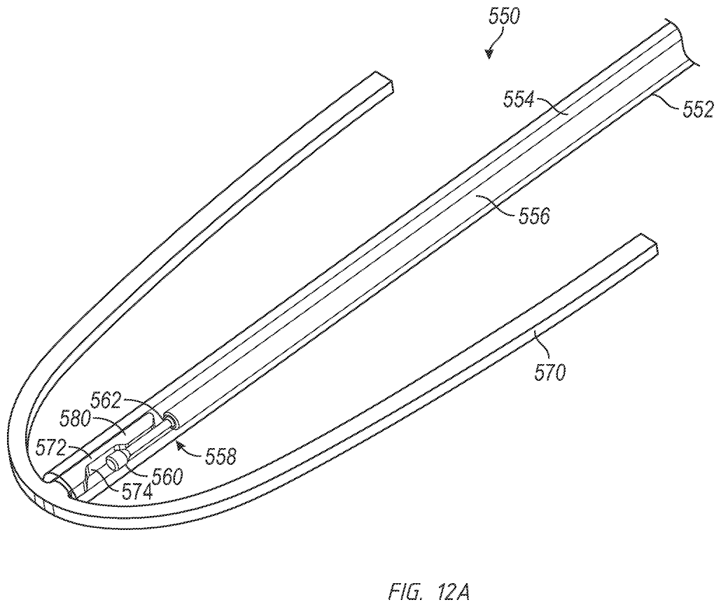

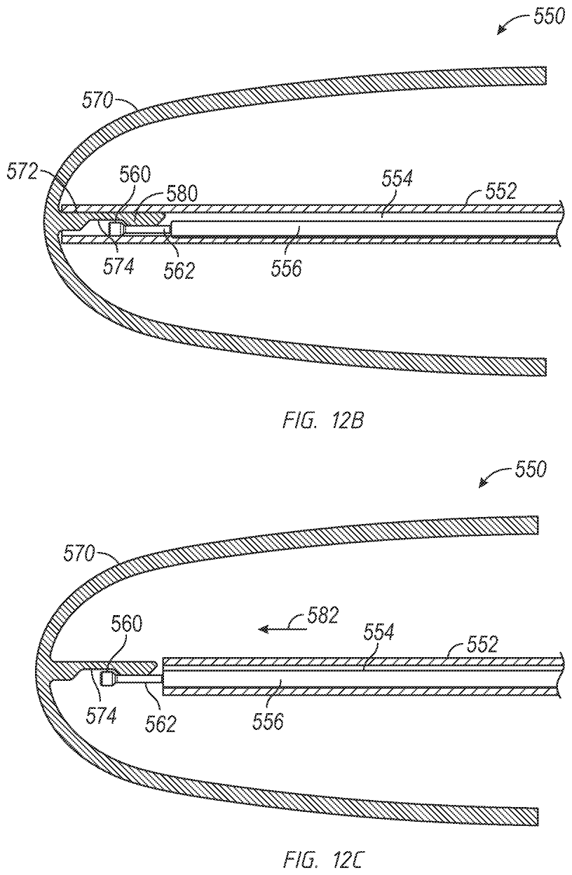

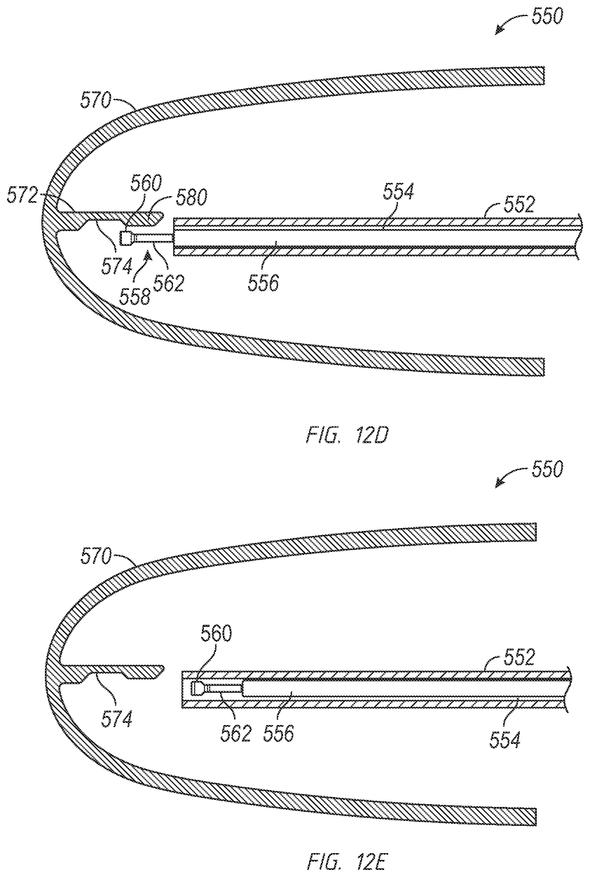

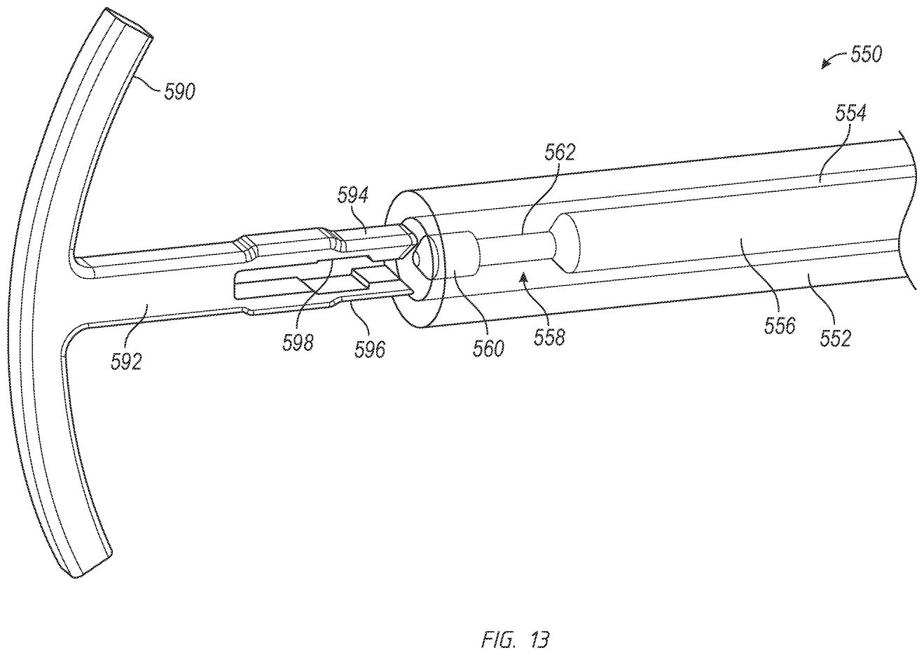

For example, as shown in FIGS. 12A-13, the grasper mechanism 550 can comprise a tubular member 552 having a lumen 554 wherein an engagement wire 556 can be positioned. The engagement wire 556 can comprise a distal end segment 558, a bulbus end portion 560, and a reduced profile section 562.

The reduced profile section 562 can comprise a reduced diameter relative to an adjacent proximal section of the engagement wire 556. Further, the bulbus end portion 560 can comprise a generally cylindrical component attached to the reduced profile section 562 that has a larger diameter than the reduced profile section 562. However, in accordance with some embodiments, the bulbus end portion 560 and the reduced profile section 562 can be configured as a notch or slot formed in a distal portion of the engagement wire 556 that is compatible with or configured to engage with a respective protrusion or component of the anchor tab of the valve anchor.

With regard to the embodiment illustrated in FIGS. 12A-12E, the grasper mechanism 550 can engage with a valve anchor 570 having an anchor tab 572 that comprises a notch portion 574. The notch portion can be configured to receive at least a portion of the bulbous end portion 560 of the engagement wire 556 therein. Further, and inner diameter of the lumen 554 can be sized to permit a combined crossing profile or outer profile of the anchor tab 572 and the distal end segment 558 of the engagement wire 556 to fit within the lumen 554 when they longitudinally overlap with each other, as shown in FIG. 12A. Moreover, when longitudinally overlapping, the bulbus end portion 560 of the engagement wire 556 will be constrained against or within the notch portion 574 of the anchor tab 572 and the reduced profile section 562 will generally constrain an enlarged end portion 580 of the anchor tab 572 such that the enlarged end portion 580 and the distal end segment longitudinally overlap with each other. In this manner, the anchor tab 572 and the reduced profile section 562 are constrained radially such that corresponding structures are actually engaged with each other and securely retain the anchor tab 572 within the lumen 554 of the tubular member 552.

FIGS. 12B-12E illustrate the stepwise release and disengagement of the anchor tab 572 from within the lumen 554 of the tubular member 552. As shown in FIG. 12B, the anchor tab 572 is engaged and secured within the lumen 554 in a first position in which the bulbous end portion 560 longitudinally overlaps with the notch portion 574 and the enlarged end portion 580 longitudinally overlaps with the reduced profile section 562. However, as the engagement wire 556 is moved distally in the direction shown by arrow 582 relative to the tubular member 552, the radial constraint of the inner surface of the lumen 554 of the tubular member 552 is removed, as shown in FIG. 12C. Once the radial constraint created by the tubular member 552 is removed, the anchor tab 572 and the distal end segment 558 of the engagement wire 556 can be radially separated, as shown in FIG. 12D.

Thereafter, as shown in FIG. 12E, the engagement wire 556 can be proximally withdrawn relative to and into the lumen 554 of the tubular member 552. The valve anchor 570 can then expand freely into apposition at the target site.

FIG. 13 illustrates another embodiment in which a valve anchor 590 comprises an anchor tab 592 having opposing elongate segments 594, 596 that form or define a gap 598 between. Similar to the embodiment discussed above with respect to FIGS. 12A-12E, the gap 598 can be configured to include corresponding recesses and protrusions that generally correspond with the shape of the bulbous end portion 560 and the reduced profile section 562 of the engagement wire 556. As described above with respect to FIGS. 12A-12E, the discussion of which is incorporated herein by reference and will not be repeated herein for brevity, the anchor tab 592 can longitudinally overlap with distal end segment 558 of the engagement wire 556 within the lumen 554 of the tubular member 552. In such a position, the anchor tab 592 will be securely engaged with the engagement wire 556 and prevented from disengagement from the grasper mechanism 550. Further, as shown and discussed with respect to the steps illustrated in FIGS. 12B-12E, the anchor tab 592 can similarly be pushed out of the lumen 554 and released from engagement with engagement wire 556.

The embodiments shown in FIGS. 12A-13 illustrate additional features or components of the anchor tab and engagement wire that permit a simplified structure to leverage an interference fit with the engagement wire and the anchor tab within the lumen of the tubular member of the grasper mechanism. Further, in accordance some embodiments, an advantage in some embodiments of the anchor tab and wire arrangement shown in FIG. 13 is that each grasper connection can be made individually via a temporary engagement by simply pressing the anchor tab onto the wire or grasper mechanism until the two notched legs snap over the bulbous end portion 560. Once all three connections are made, the handle can be actuated to pull all three wires into the respective tubular members, thus having the widest portion of the anchor tab 592 effectively locking or enclosing the bulbous end portion 560 in engagement within the gap 598. Such an arrangement can simplify the connection by providing a temporary connection at each wire, followed by a common locking actuation during assembly. This can advantageously provide a simplified mechanical operation and easier assembly.

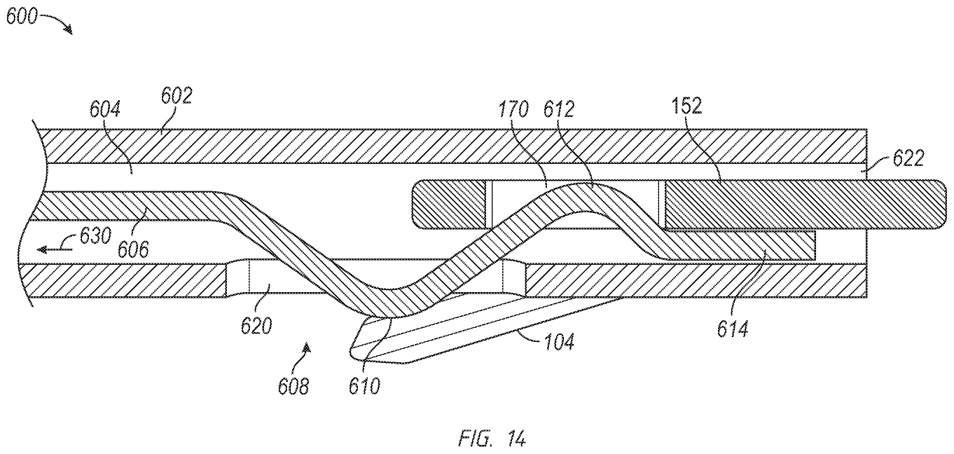

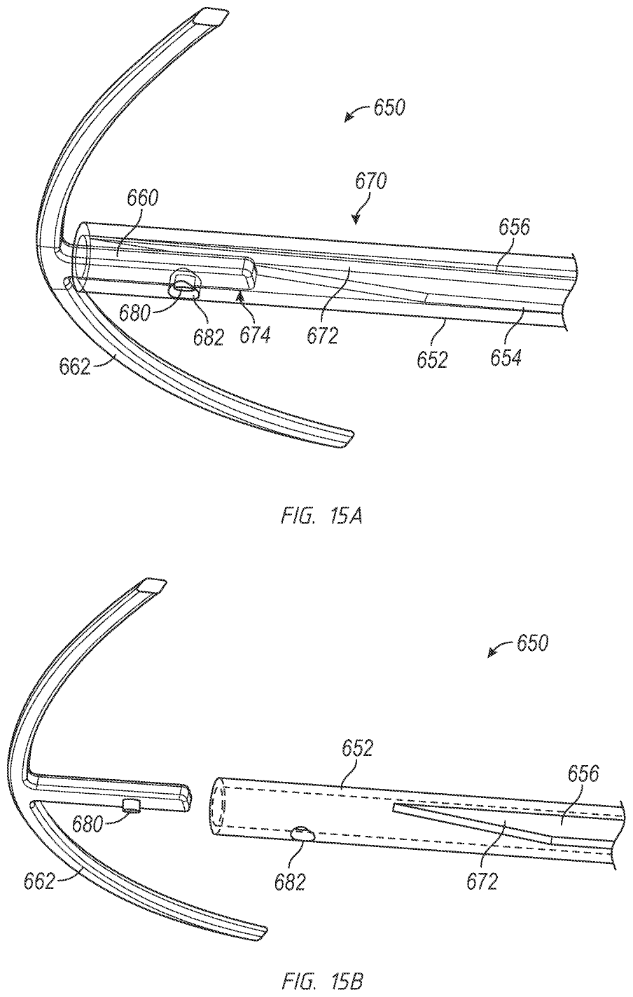

Furthermore, some embodiments can optionally comprise other interference fit mechanisms that radially engage with the anchor tab of the valve anchor within the lumen of the tubular member. Proximal withdrawal of such structures, which can be which can be incorporated into the engagement wire or whose function can be performed by the engagement wire to create some radial constriction or interference, are shown in FIGS. 14-15B, for example. These additional embodiments illustrate principles of other components and configurations that can be prepared using aspects of some embodiments.

For example, FIG. 14 illustrates an embodiment of a grasper mechanism 600 comprising a tubular member 602 having a lumen 604 and an engagement wire 606 disposed within the lumen 604. The engagement wire 606 can comprise a zigzag section 608 at a distal end portion thereof. The zigzag section 608 can have a configuration whereby the engagement wire 606 deviates from a generally straight configuration to have one or more bands along the length of the wire 606 in the zigzag section 608.

For example, the zigzag section 608 can be formed having at least two, at least three, or more bends. As illustrated in FIG. 14, the zigzag section 608 can comprise four bends whereby the engagement wire 606 deviates from a generally straight shape or configuration. The bends can form first and second bends 610, 612, and the engagement wire 606 can further comprise a distal section 614 that extends distally beyond the second bend 612 within the lumen 604.

In accordance with some embodiments, the tubular member 602 can comprise a slot 620 and an end opening 622. When coupled to a valve anchor, such as valve anchor 104, the anchor tab 152 of the valve anchor 104 can be inserted into the end opening 622 of the tubular member and engaged with a portion of the engagement wire 606.

For example, as illustrated, the slot 170 of the anchor tab 152 can receive at least a portion of the second bend 612 and longitudinally overlap with the distal section 614 of the engagement wire 606. In this manner, the anchor tab 152 can be radially and longitudinally constrained at least in part due to the protuberance of the second bend 612 which radially inserts into the slot 170 and is received therein to form an interference fit between the anchor tab 152 and the distal section 614 of the engagement wire 606 within the lumen 604. Thus, the size of the lumen 604 can be sufficient to receive both the profile or diameter of the engagement wire 606 and the profile of the anchor tab 152 such that they longitudinally overlap each other, but due to the insertion of the second bend 612 into the slot 170, the anchor tab 152 will not be able to exit the end opening 622 in the position illustrated in FIG. 14.

Further, because the first bend 610 can insert or be received through the slot 620 of the tubular member 602, the engagement wire 606 will tend to be longitudinally or actually constrained relative to the tubular member 602.

However, in accordance with some embodiments, the engagement wire 606 can be proximately retracted using sufficient proximal force, thereby pulling upwardly on the first bend 610 to straighten the first bend 610 and exerting a similar pulling/straightening force on the second bend 612. Accordingly, as the zigzag section 608 is pulled proximately, the first and second bends 610, 612 can be longitudinally straightened, thereby removing the interference fit between the anchor tab 152 and the second bend 612 of the engagement wire 606 within the lumen 604. Continued proximal retraction of the engagement wire will cause the zigzag section 608 to be pulled to a location proximal to the slot 620 in the direction illustrated by the arrow 630. As the anchor tab 152 is permitted to exit the end opening 622, the valve anchor 104 and the grasper mechanism 600 will be separated and disengage from each other, thereby permitting the valve anchor 104 to expand into apposition with the native valve tissue at the target site.

FIGS. 15A and 15B illustrate another embodiment that utilizes cross-sectional interference fits to cause engagement between an anchor tab of a valve anchor and a grasper mechanism, according to some embodiments.

Referring to FIG. 15A, a grasper mechanism 650 can comprise a tubular member 652 having a lumen 654 and an engagement wire 656. The engagement wire 656 can comprise a tapering shape, such as a wedge-shaped, that tapers in a distal direction such that the engagement wire 656 can be longitudinally overlapped with an anchor tab 660 of a valve anchor 662. In a first position 670, a reduced profile section 672 of the engagement wire 656 can be urged into radial engagement so as to form an interference fit along a proximal portion 674 of the anchor tab 660. The reduced profile section 672 can comprise a wedge or a conical shape, according to some embodiments.

As will be appreciated by personal skill in the art, as the reduced profile section 672 is urged distally relative to the anchor tab, the cross-sectional profile of the reduced profile section 672 will increase until the total cross-sectional area of the anchor tab 660 and the reduced profile section 672 matches an inner profile of the lumen 654 of the tubular member 652. In such a position, a protrusion 680 of the anchor tab 660, which extends through a side aperture 682 of the tubular member 652 can be fully extended into the side aperture 682. The presence of the protrusion 680 and its insertion into the side aperture 682 can tend to ensure that the anchor tab 660 is unable to slide or translate relative to the tubular member 652, thereby constraining relative longitudinal movement thereof and retaining the anchor tab 660 within the lumen 654 of the grasper mechanism 650.

However, as illustrated in FIG. 15B, the engagement wire 656 can be proximately withdrawn into the lumen 654, thereby proximately withdrawing the reduced profile section 672 away from the proximal portion 674 of the anchor tab 660. As the reduced profile section 672 is proximally withdrawn away from the proximal portion 674 of the anchor tab 660, the proximal portion 674 of the anchor tab 660 can radially move relative to the sidewall or inner surface of the lumen 654, thereby permitting the protrusion 680 to exit the aperture 682. As this occurs, the anchor tab 660 can be dislodged and separated from the tubular member 652 after the protrusion 680 separates from the aperture 682. Thus, FIG. 15B illustrates the separation of the valve anchor 662 from the tubular member 652.

Accordingly, the simplicity of the designs and FIGS. 14-15B can provide certainty and simplified action of the grasper mechanism, according to some embodiments. The simple proximal retraction of the engagement wire and subsequent elimination of cross-sectional interference between the anchor tab and the engagement wire can allow a simple separation and release of the valve anchor from the grasper mechanism in operating the deployment device.

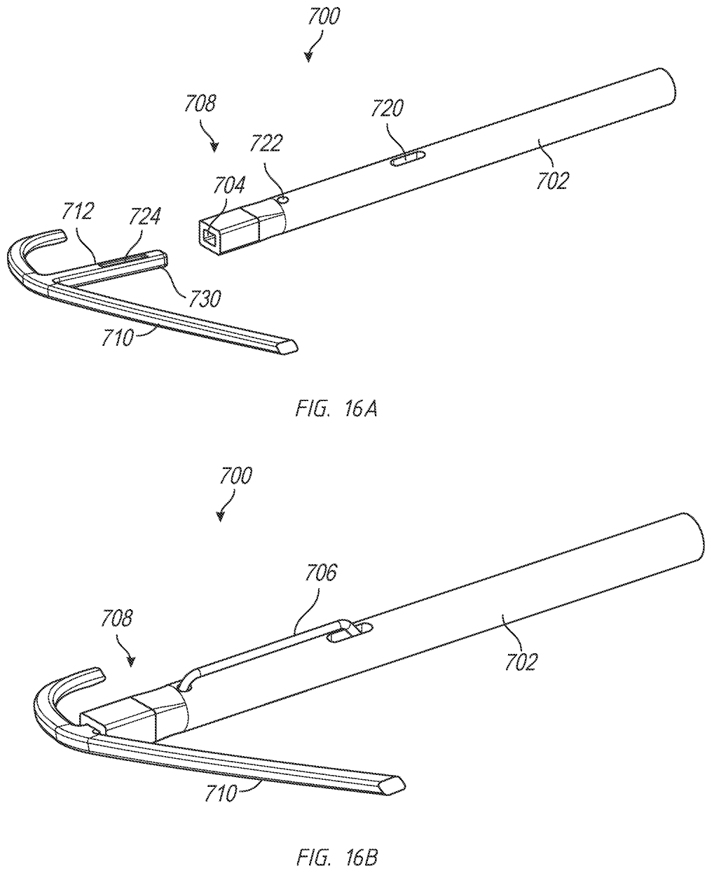

In accordance with yet another embodiment disclosed herein, FIGS. 16A and 16B illustrate another grasper mechanism, according to some embodiments. FIG. 16A illustrates a grasper mechanism 700 that can comprise a tubular member 702 having a lumen 704 configured to receive an engagement wire 706 and a distal end section 708 configured to limit a degree of freedom of movement of a valve anchor 710 when coupled thereto.

As in other embodiments disclosed herein, the lumen 704 of the grasper mechanism 700 can receive a portion of the valve anchor 710, such as an anchor tab 712 of the valve anchor 710. Then, once the anchor tab 712 is in place within the lumen 704, the engagement wire 706 can be looped through proximal and distal apertures 720, 722 to engage a slot or aperture 724 of the anchor tab 712, as shown in FIG. 16B. The valve anchor 710 will be securely engaged relative to the grasper mechanism 700, as with other embodiments disclosed herein that limit relative axial or longitudinal movement between the anchor tab 712 of the valve anchor 710 and the grasper mechanism 700.

However, in accordance with some embodiments, the distal end section 708 and/or the lumen 704 can advantageously be configured to limit an additional degree of freedom of movement of the anchor tab 712 when engaged therewith. In this manner, some embodiments can tend to provide a more rigid interconnection between the grasper device.

For example, the lumen 704 can restrict rotational movement of the anchor tab 712 relative to and within the lumen 704. This can be achieved by forming the anchor tab 712 with a polygonal cross-section, such as a rectangle, square, or triangle, or other shape having one or more protruding corners, flanges, or tabs that can engage with a corresponding structure or shape of the of the anchor tab 712 in order to restrict rotational movement of the anchor tab 712 relative to the lumen 704. By restricting rotational movement therebetween, the valve anchor 710 will tend to move in unison with the grasper mechanisms 700 of the grasper device, thereby creating more control and precision when placing the valve anchor 710.

Accordingly, in accordance with some embodiments, the anchor tab 712 can have a profile or cross-sectional shape that is configured to mate with a corresponding profile or cross-sectional shape of the lumen 704. The cross-sectional shape of the anchor tab can comprise a rectangle having beveled corners 730 that permit the flat surfaces formed by the sides of the rectangular shape to provide the rotational constraint while the beveled corners 730 allow the anchor tab 712 to be able to slide within the lumen 704 with some case.

As illustrated generally in FIG. 16A, in accordance with some embodiments, the beveled corners 730 can comprise a radius of curvature that is between about 10% and about 50% of the distance of a side of the rectangular profile of the anchor tab 712, such as about 1/7, ⅙, ⅕, ¼, or about ⅓ of the distance of a side of the rectangular profile of the anchor tab 712. Further, the rectangular profile of the anchor tab 712 can define about a length ratio of adjacent sides of about 2:3, 1:2, 1:3, 1:4, or 1:5.

In some embodiments, the rectangular profile of the anchor tab 712 can provide a tab width that is at least 20%, 30%, 40%, 50%, 60%, 70%, 80%, 90%, or hundred percent larger than a width of the slot 724 extending through the anchor tab 712.

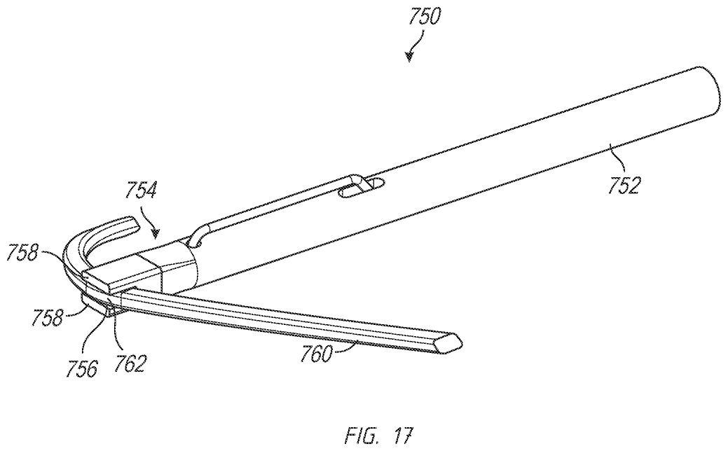

Optionally, as shown in FIG. 17, the distal end section can be formed to include an anchor slot or bay into which the anchor can be at least partially received to provide a further rotational restraint between the grasper mechanism and the valve anchor.

As illustrated, the grasper mechanism 750 can comprise a tubular member 752 that includes a distal end section 754. The distal end section 754 comprises an anchor slot or bay 756 that provides a gap or space between opposing flanges 758. The valve anchor 760 can be received within the anchor slot 756 and fitted into the gap between the opposing flanges 758. The generally flat profile of the flanges 758 fits against top and bottom edges of the valve anchor 760 along a width or sideways length of the anchor slot 756 to constrain rotational movement therebetween.

Thus, in some embodiments, with the anchor tab (not shown) of the valve anchor 760 being received into a lumen of the tubular member 752, the anchor slot 756 can further engage a base 762 of the anchor tab 760 and provide a restraint against relative rotational movement between the anchor 760 and the tubular member 752 about a longitudinal axis of the tubular member 752. Such embodiments can be implemented in combination with or separately from the anchor tab having a specific cross-sectional profile to provide a rotational constraint and engagement with the lumen of the tubular member, as discussed above with respect FIGS. 16A and 16B.

FIGS. 16A-17 show that in some embodiments, the tubular member can comprise a round (e.g., circular) outer profile along the entirety of its length or at least a portion of its length. Further, some embodiments can have a non-circular outer profile along the entirety of its length or at least a portion of its length.

In addition, various embodiments throughout the present disclosure illustrate that the lumen of the tubular member can have a generally round (e.g., circular) cross sectional inner profile or luminal profile. FIGS. 16A and 16B illustrate that the tubular member can comprise a non-round (e.g., non-circular) luminal profile along at least a portion of its length. As noted above, the lumen can comprise a polygonal cross-section, such as a rectangle, square, or triangle, or other shape having one or more protruding corners, flanges, or tabs that can engage with a corresponding structure or shape of the anchor tab. Such a non-round luminal profile can be formed along the entirety of the length of the tubular member or at least a portion of its length.

The shape of at least a portion of the outer profile or the luminal profile can be formed or adjusted via an extrusion process or via post-extrusion heating and deformation to change the outer profile or luminal profile, as desired, using end-portion-forming tools onto which the tubular member is pressed to change the outer profile and/or luminal profile.

Further, in some embodiments, a round or non-round outer profile can be along the entirety or only a portion of the length of the tubular member while a round or non-round luminal profile extends along the entirety or only a portion of the length of the tubular member. Thus, the tubular member can have a round outer profile that changes to a non-round outer profile, along with a non-round luminal profile.

Accordingly, in accordance with some embodiments, a clinician can manipulate the valve anchor by engaging or coupling a grasper with an anchor tab of a valve anchor. The grasper and the anchor tab can be restricted from relative longitudinal, rotational, and/or radial movement by enclosing or interconnecting the grasper and the anchor tab alongside each other, such as within or against a tubular enclosure. In order to disengage the grasper and the anchor tab, the clinician can withdraw or advance an engagement wire disposed within the grasper. This relative movement can cause or permit a dislocation or separation between the grasper and the anchor tab. Once separated, the valve anchor will tend to expand radially outwardly, thereby disengaging the anchor tab from the grasper. Thereafter, the grasper can be retracted or withdrawn into the tubular enclosure.

### Illustration of Subject Technology as Clauses

Various examples of aspects of the disclosure are described as clause sets having numbered clauses (1, 2, 3, etc.) for convenience. These are provided as examples, and do not limit the subject technology. Identifications of the figures and reference numbers are provided below merely as examples and for illustrative purposes, and the clauses are not limited by those identifications.

Clause 1. A prosthetic heart valve delivery device comprising: a docking area defining a longitudinal axis; and an engagement wire extending along the longitudinal axis, the engagement wire comprising a first section, a second section, and a looped section interposed between the first section of the second section along the engagement wire, the engagement wire having (i) an engaged position in which the looped section extends through the docking area and the second section at least partially longitudinally overlaps with the first section to facilitate engagement between the delivery device and a heart valve prosthesis at the docking area and (ii) a disengaged position in which the second section is positioned distal to the looped section and the first section to permit disengagement of the heart valve prosthesis from the docking area.

Clause 2. The delivery device of any of the preceding Clauses, further comprising a tubular member having a lumen that at least partially defines the docking area.

Clause 3. The delivery device of Clause 2, wherein the engagement wire extends through the lumen.

Clause 4. The delivery device of any of Clauses 2 or 3, wherein the docking area is configured to receive a protrusion or tab of the heart valve prosthesis therein.

Clause 5. The delivery device of any of Clauses 2 to 4, wherein the tubular member comprises at least one aperture extending through a sidewall thereof to the lumen thereof, and wherein in the engaged position, the engagement wire is positioned within the lumen and extends through the aperture.

Clause 6. The delivery device of any of Clauses 2 to 5, wherein the tubular member comprises a distalmost set of apertures extending through a sidewall of the tubular member toward the lumen thereof, the engagement wire extending through the distalmost set of apertures for facilitating engagement between the delivery device and the heart valve prosthesis at the docking area.

Clause 7. The delivery device of any of the preceding Clauses, further comprising a tubular member having a lumen that at least partially defines the docking area, the tubular member comprising four apertures extending from an outer surface of the tubular member to the lumen, wherein: the engagement wire is disposed through a first aperture and a second aperture such that the second section of the engagement wire is disposed outside of the lumen; the engagement wire is disposed through the second aperture and a third aperture such that a second segment of the engagement wire extends through the docking area within the lumen; the engagement wire is disposed through the third aperture and a fourth aperture such that third segment of the engagement wire is disposed outside the lumen; and the engagement wire is disposed through the fourth aperture such that a fourth segment of the engagement wire is disposed outside the lumen.

Clause 8. The delivery device of any of the preceding Clauses, further comprising a tubular member having a lumen that at least partially defines the docking area, the tubular member comprising four apertures extending from an outer surface of the tubular member to the lumen, wherein proximal and distal segments of the engagement wire are disposed within the lumen, engagement wire passing through the four apertures to form a loop segment that extends through the docking area within the lumen.

Clause 9. The delivery device of any of the preceding Clauses, further comprising a tubular member having a lumen that at least partially defines the docking area, the tubular member comprising first, second, third, and fourth of apertures extending from an outer surface of the tubular member to the lumen, the first and fourth apertures each having a longitudinal axis that extends transverse relative to a longitudinal axis of the tubular member, the second and third apertures each having a longitudinal axis that extends substantially perpendicular relative to a longitudinal axis of the tubular member.

Clause 10. The delivery device of Clause 9, wherein the second and third apertures are disposed at approximately equal longitudinal positions along the longitudinal axis of the tubular member.

Clause 11. The delivery device of any of Clauses 9 or 10, wherein the first aperture is interposed between the fourth aperture and the second and third apertures along the longitudinal axis of the tubular member.

Clause 12. The delivery device of any of Clauses 9 to 11, wherein the first aperture is longitudinally offset from the second, third, and fourth apertures along the longitudinal axis of the tubular member.

Clause 13. The delivery device of any of the preceding Clauses, further comprising a tubular member having a lumen that at least partially defines the docking area, the tubular member comprising a plurality of apertures extending from an outer surface of the tubular member to the lumen, the engagement wire extending through the lumen and the plurality of apertures.

Clause 14. The delivery device of Clause 13, further wherein at least one aperture is longitudinally offset from another aperture along the longitudinal axis of the tubular member.

Clause 15. The delivery device of any of the preceding Clauses, further comprising a tubular member having a lumen that at least partially defines the docking area, an open end that permits access to the docking area, and a plurality of apertures extending from an outer surface of the tubular member to the lumen for permitting the engagement wire to extend out of and into the lumen and across the docking area to engage a portion of the heart valve prosthesis extending into the open end of the tubular member.

Clause 16. The delivery device of any of the preceding Clauses, further comprising three tubular members that form respective docking areas for engaging the valve therewith.

Clause 17. The delivery device of Clause 16, further comprising a proximal sheath wherein the three tubular members extend, the proximal sheath being retractable to permit expansion of the heart valve prosthesis and three tubular members prior to disengagement of the delivery device from the heart valve prosthesis.

Clause 18. The delivery device of any of the preceding Clauses, wherein the docking area comprises an enclosure having an opening for receiving a portion of the heart valve prosthesis to permit coupling between the heart valve prosthesis and the delivery device.

Clause 19. The delivery device of any of the preceding Clauses, further comprising a tubular member having a lumen and a distalmost pair of apertures that extend from an outer surface of the tubular member into the lumen, the distalmost pair of apertures being diametrically opposed and positioned at a first longitudinal position along a longitudinal axis of the tubular member, the engagement wire extending through the distalmost pair of apertures into the lumen and across the docking area for permitting engagement with the heart valve prosthesis.

Clause 20. The delivery device of Clause 19, wherein the first longitudinal position is set at a first distance from an end of the tubular member, wherein the heart valve prosthesis comprises an elongate tab having a base and an aperture extending through the elongate tab, wherein the first distance is about equal to a distance from an edge of the aperture and the base for permitting a close fit and minimizing relative movement between the elongate tab and the tubular member in the engaged position.

Clause 21. A prosthetic heart valve delivery device for delivering a heart valve prosthesis, the device comprising: a core member defining a longitudinal axis; a proximal sheath extending over the core member; and a plurality of grasper mechanisms extending along the longitudinal axis within the proximal sheath for engaging the heart valve prosthesis, each grasper mechanism comprising a longitudinal axis, an elongate docking area extending along the longitudinal axis, and an engagement wire extending along the grasper mechanism, the engagement wire comprising a proximal section and a looped section, the looped section extending through the docking area in a direct transverse relative to the longitudinal axis to facilitate engagement between the grasper mechanism and the heart valve prosthesis in an engaged configuration, the engagement wire being proximally retractable to proximally withdraw the looped section from the docking area for disengaging the grasper mechanism from the heart valve prosthesis in a disengaged configuration.

Clause 22. The delivery device of Clause 21, wherein the plurality of grasper mechanisms comprises three grasper mechanisms.

Clause 23. The delivery device of any of Clauses 21 or 22, wherein each grasper mechanism comprises a tubular member wherein the docking area is disposed.

Clause 24. The delivery device of Clause 23, wherein the tubular member comprises a plurality of apertures extending through a sidewall of the tubular member to a lumen thereof, the plurality of apertures being configured to permit passage of the engagement wire therethrough.

Clause 25. The delivery device of Clause 24, wherein the plurality of apertures comprises a pair of opposing apertures positioned at approximately the same axial position relative to a longitudinal axis of the tubular member, the opposing apertures being substantially diametrically opposed from each other.

Clause 26. The delivery device of Clause 25, wherein the engagement wire passes through the opposing apertures to cross the docking area in the engaged configuration.

Clause 27. The delivery device of any of Clauses 25 or 26, wherein the plurality of apertures further comprises at least one proximal aperture in the tubular member, proximal to the pair of opposing apertures, the at least one proximal aperture being configured to permit the engagement wire to extend from the lumen and to a location outside of the lumen along the tubular member.

Clause 28. The delivery device of any of Clauses 24 to 27, wherein the plurality of apertures comprises four apertures.

Clause 29. The delivery device of any of Clauses 24 to 28, wherein the plurality of apertures comprises five apertures.

Clause 30. The delivery device of Clause 23, wherein the tubular member comprises an elongate receptacle disposed along a sidewall of the tubular member.

Clause 31. The delivery device of Clause 30, wherein the receptacle extends at least partially into the sidewall, the receptacle having an axial length that is greater than a width thereof.

Clause 32. The delivery device of any of Clauses 30 or 31, wherein the receptacle comprises a shoulder area defined by a recess along a length of the receptacle that is configured to receive a portion of the heart valve prosthesis therein, the shoulder area restricting at least one degree of freedom of movement of the portion of the heart valve prosthesis.

Clause 33. The delivery device of Clause 32, wherein the receptacle comprises a pair of opposing rail portions extending at least partially along the length of the receptacle, the opposing rail portions being configured to laterally restrict motion of at least a portion of the heart valve prosthesis.

Clause 34. The delivery device of Clause 33, wherein in the engaged configuration, the looped section of the engagement wire extends from a lumen of the tubular member through the receptacle to outside of the lumen for engaging with the heart valve prosthesis.

Clause 35. The delivery device of Clause 34, wherein the tubular member comprises a pair of opposing apertures extending through a sidewall of the tubular member from the lumen thereof, the pair of opposing apertures being generally diametrically opposed relative to each other around the tubular member, wherein the looped section of the engagement wire extends through the receptacle, through the pair of opposing apertures, through the lumen, and to opposing outer surfaces of the tubular member.

Clause 36. The delivery device of Clause 35, wherein the tubular member comprises proximal apertures extending through the sidewall of the tubular member, wherein in the engaged configuration, the engagement wire extends, in sequence, out of the lumen through a proximal aperture, into the lumen through another proximal aperture, through the receptacle and docking area to a location outside of the lumen, through the pair of opposing apertures to pass through the lumen and to outside the lumen, and into yet another proximal aperture into the lumen.

Clause 37. The delivery device of any of Clauses 34 to 36, wherein the tubular member comprises an end cover member disposed that a distal end of the tubular member, the end cover member being configured to be coupled to a distal end of the engagement wire for securing the engagement wire extending from the lumen over the receptacle to facilitate engagement with the heart valve prosthesis in the engaged configuration.

Clause 38. The delivery device of Clause 37, wherein the end cover member is releasably coupled to the end cover member.

Clause 39. The delivery device of Clause 38, wherein the end cover member is adhesively or mechanically coupled to the end cover member.

Clause 40. The delivery device of any of Clauses 21 to 39, wherein the engagement wire comprises a pull wire and a loop wire, the loop wire having an aperture structure wherethrough the pull wire can extend in the engaged configuration, wherein the pull wire is proximally retractable to disengage from the aperture structure to permit separation of the pull wire from the loop wire, the pull wire and the loop wire being proximally retractable relative to the docking area to permit disengagement of the grasper mechanism from the heart valve prosthesis.

Clause 41. The delivery device of Clause 40, wherein each grasper mechanism comprises a tubular member wherein the docking area is disposed, the tubular member comprising a lumen configured to receive the pull wire and the loop wire upon proximal retraction of the pull wire and the loop wire.

Clause 42. The delivery device of Clause 41, wherein the tubular member comprises a pair of opposing apertures extending through a sidewall of the tubular member from the lumen thereof, the pair of opposing apertures being generally diametrically opposed relative to each other around the tubular member, wherein the loop wire extends through the pair of opposing apertures through the docking area, the loop wire being engaged by the pull wire within the lumen of the tubular member.

Clause 43. A prosthetic heart valve delivery device for delivering a heart valve prosthesis, the device comprising: a grasper mechanism comprising a longitudinal axis, a tubular member having a lumen, a docking aperture extending through a sidewall of the tubular member into the lumen, and an engagement wire extending through the lumen toward the docking aperture, the engagement wire being configured to pass through and engage a slot or aperture of a clasper tang of the heart valve prosthesis, the engagement wire extending generally parallel relative to the longitudinal axis and the clasper tang extending generally transverse relative to the longitudinal axis to facilitate engagement between the grasper mechanism and the heart valve prosthesis in an engaged configuration, the engagement wire being proximally retractable from the slot or aperture of the clasper tang for disengaging the grasper mechanism from the heart valve prosthesis in a disengaged configuration.

Clause 44. The delivery device of Clause 43, wherein the engagement wire comprises a straight wire.

Clause 45. The delivery device of any of Clauses 43 or 44, wherein the docking aperture extends in a direction transverse relative to the longitudinal axis along the sidewall of the tubular member.

Clause 46. The delivery device of any of Clauses 43 to 45, wherein the device comprises a plurality of grasper mechanisms, wherein the docking aperture of each grasper mechanism faces in a direction away from other grasper mechanisms.

Clause 47. A prosthetic heart valve delivery device comprising: a grasper mechanism comprising a longitudinal axis, a tubular member having a lumen and an open end, and an engagement wire extending through the lumen toward the open end, the engagement wire having a distal end segment having a protrusion or recess configured to abut a respective recess or protrusion of a clasper tang of a heart valve prosthesis to form a component coupling, wherein: in an engaged configuration, the distal end segment and the clasper tang longitudinally overlap within the lumen and collectively define a cross-sectional profile that is less than an inner diameter of the lumen such that movement of the distal end segment and the clasper tang is radially constrained by the lumen to restrict movement of the distal end segment relative to the clasper tang, and the distal end segment of the engagement wire can be moved distally out of the lumen to permit the distal end segment and the clasper tang to become radially displaced from each other to a disengaged configuration to permit the clasper tang to move freely away from the engagement wire for disengaging the heart valve prosthesis from the grasper mechanism.

Clause 48. The delivery device of Clause 47, wherein the distal end segment of the engagement wire comprises an elongate prong having an enlarged end portion and a notch portion, the notch portion being proximal to the enlarged end portion and having a smaller cross-sectional profile than the enlarged end portion.

Clause 49. The delivery device of any of Clauses 47 or 48, wherein the distal end segment of the engagement wire comprises a pair of elongate prongs, each elongate prong having an enlarged end portion and a notch portion, the notch portion being proximal to the enlarged end portion and having a smaller cross-sectional profile than the enlarged end portion.

Clause 50. The delivery device of Clause 49, wherein each elongate prong comprises a lateral bump, each lateral bump extending radially from a surface of the elongate prong in a direction away from both elongate prongs, the lateral bumps being configured to contact an inner surface of the lumen for maintaining engagement between the distal end segment and the clasper tang of the heart valve prosthesis.

Clause 51. The delivery device of any of Clauses 47 to 50, wherein the distal end segment comprises dual opposing hooks configured to engage the clasper tang of the heart valve prosthesis.

Clause 52. A prosthetic heart valve system comprising the delivery device of any of Clauses 47 to 50 and a heart valve prosthesis comprising a clasper tang, the clasper tang having an elongate prong having an enlarged end portion and a notch portion, the notch portion being proximal to the enlarged end portion and having a smaller cross-sectional profile than the enlarged end portion.

Clause 53. A prosthetic heart valve system comprising the delivery device of any of Clauses 47 to 50 and a heart valve prosthesis comprising a clasper tang, the clasper tang having a pair of elongate prongs, each elongate prong having an enlarged end portion and a notch portion, the notch portion being proximal to the enlarged end portion and having a smaller cross-sectional profile than the enlarged end portion.

Clause 54. The system of Clause 53, wherein each elongate prong comprises a lateral bump, each lateral bump extending radially from a surface of the elongate prong in a direction away from both elongate prongs, the lateral bumps being configured to contact an inner surface of the lumen for maintaining engagement between the distal end segment and the clasper tang of the heart valve prosthesis.

Clause 55. The system of any of Clauses 53 or 54, wherein the clasper tang comprises dual opposing hooks configured to engage the distal end segment of the engagement wire in the engaged configuration.

Clause 56. A prosthetic heart valve delivery device comprising: a grasper mechanism comprising: a tubular member having a lumen, a side aperture extending from the lumen to an outer surface of the tubular member, and an open end; and an engagement wire extending through the lumen toward the open end, the engagement wire having a distal end segment configured to at least partially longitudinally overlap with a clasper tang of a heart valve prosthesis positioned within the lumen; wherein (i) in an engaged position, the distal end segment is that a first position that longitudinally overlaps with the clasper tang within the lumen to create an interference fit within the lumen to causes an engagement structure coupled to the engagement wire or the clasper tang to protrude into the side aperture of the tubular member and thereby restrict longitudinal movement of the clasper tang relative to the tubular member, and wherein (ii) in a disengaged position, the distal end segment is positioned at a second position, proximal to the first position, in which the distal end segment and the clasper tang release the interference fit and permit the engagement structure to withdraw from the side aperture to thereby permit longitudinal movement of the clasper tang relative to the tubular member.

Clause 57. The delivery device of Clause 56, wherein the distal end segment of the engagement wire comprises a zigzag section having first and second bends, wherein in the engaged position, the first bend is configured to be received within the side aperture of the tubular member and the second bend is configured to engage with an aperture of the clasper tang.

Clause 58. The delivery device of Clause 57, wherein the distal end segment comprises a straight distalmost section, distal to the first and second bends, configured to longitudinally overlap with the clasper tang within the lumen in the engaged position to assist in creating the interference fit.

Clause 59. The delivery device of any of Clauses 56 to 58, wherein the distal end segment of the engagement wire comprises a tapering cross-sectional profile that decreases in a distal direction.

Clause 60. The delivery device of Clause 59, wherein the distal end segment comprises a wedge section that decreases in a distal direction.

Clause 61. The delivery device of any of Clauses 59 or 60, wherein the distal end segment comprises a flat surface configured to abut the clasper tang.

Clause 62. A prosthetic heart valve system comprising the delivery device of Clause 56 and a heart valve prosthesis having a clasper tang, wherein the clasper tang comprises an elongate body having a protrusion extending in a direction transverse relative to a longitudinal access of the elongate body, the protrusion being configured to protrude into the side aperture of the tubular member to restrict longitudinal movement of the clasper tang relative to the tubular member when the clasper tang is in the engaged position with the engagement wire of the delivery device.

Clause 63. A prosthetic heart valve delivery device comprising any of the features recited in any of the preceding Clauses.

Clause 64. A prosthetic heart valve system comprising a heart valve prosthesis and a prostatic heart valve delivery device comprising any of the features recited in Clauses 1 to 62.

Clause 65. A method of assembling a prosthetic heart valve delivery device recited in any of the preceding Clauses.

Clause 66. A method of delivering a prosthetic heart valve delivery device recited in any of the preceding Clauses comprising advancing a heart valve prosthesis to a target location within a human heart and releasing the heart valve prosthesis from the delivery device.

Clause 67. The devices, systems, and methods recited in any of the clauses above, wherein the anchor tab comprises a cross-sectional protrusion or profile configured to engage with a corresponding cross-sectional indentation or profile of the tubular member for restricting relative rotation between the anchor tab and the tubular member when the anchor tab is received within the lumen.

Clause 68. The devices, systems, and methods recited in any of the clauses above, wherein a distal end portion of the tubular member comprises an anchor slot configured to receive a portion of the valve anchor therein for restricting relative rotation between the valve anchor and the distal end portion about a longitudinal axis of the tubular member.

### FURTHER CONSIDERATIONS

In some embodiments, any of the clauses herein may depend from any one of the independent clauses or any one of the dependent clauses. In some embodiments, any of the clauses (e.g., dependent or independent clauses) may be combined with any other one or more clauses (e.g., dependent or independent clauses). In some embodiments, a claim may include some or all of the words (e.g., steps, operations, means or components) recited in a clause, a sentence, a phrase or a paragraph. In some embodiments, a claim may include some or all of the words recited in one or more clauses, sentences, phrases or paragraphs. In some embodiments, some of the words in each of the clauses, sentences, phrases or paragraphs may be removed. In some embodiments, additional words or elements may be added to a clause, a sentence, a phrase or a paragraph. In some embodiments, the subject technology may be implemented without utilizing some of the components, elements, functions or operations described herein. In some embodiments, the subject technology may be implemented utilizing additional components, elements, functions or operations.

The foregoing description is provided to enable a person skilled in the art to practice the various configurations described herein. While the subject technology has been particularly described with reference to the various figures and configurations, it should be understood that these are for illustration purposes only and should not be taken as limiting the scope of the subject technology.

There may be many other ways to implement the subject technology. Various functions and elements described herein may be partitioned differently from those shown without departing from the scope of the subject technology. Various modifications to these configurations will be readily apparent to those skilled in the art, and generic principles defined herein may be applied to other configurations. Thus, many changes and modifications may be made to the subject technology, by one having ordinary skill in the art, without departing from the scope of the subject technology.

It is understood that the specific order or hierarchy of steps in the processes disclosed is an illustration of exemplary approaches. Based upon design preferences, it is understood that the specific order or hierarchy of steps in the processes may be rearranged. Some of the steps may be performed simultaneously. The accompanying method claims present elements of the various steps in a sample order, and are not meant to be limited to the specific order or hierarchy presented.

As used herein, the term “distal” can denote a location or direction that is away from a point of interest, such as the user or a control unit or region of the delivery system that will be used to deliver a valve prosthesis to a native valve annulus. Additionally, the term “proximal” can denote a location or direction that is closer to a point of interest, such as the user or a control unit or region of the delivery system that will be used to deliver a valve prosthesis.

As used herein, the phrase “at least one of” preceding a series of items, with the term “and” or “or” to separate any of the items, modifies the list as a whole, rather than each member of the list (i.e., each item). The phrase “at least one of” does not require selection of at least one of each item listed; rather, the phrase allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

Terms such as “top,” “bottom,” “front,” “rear” and the like as used in this disclosure should be understood as referring to an arbitrary frame of reference, rather than to the ordinary gravitational frame of reference. Thus, a top surface, a bottom surface, a front surface, and a rear surface may extend upwardly, downwardly, diagonally, or horizontally in a gravitational frame of reference.

Furthermore, to the extent that the term “include,” “have,” or the like is used in the description or the claims, such term is intended to be inclusive in a manner similar to the term “comprise” as “comprise” is interpreted when employed as a transitional word in a claim.

The word “exemplary” is used herein to mean “serving as an example, instance, or illustration.” Any embodiment described herein as “exemplary” is not necessarily to be construed as preferred or advantageous over other embodiments.

A reference to an element in the singular is not intended to mean “one and only one” unless specifically stated, but rather “one or more.” Pronouns in the masculine (e.g., his) include the feminine and neuter gender (e.g., her and its) and vice versa. The term “some” refers to one or more. Underlined and/or italicized headings and subheadings are used for convenience only, do not limit the subject technology, and are not referred to in connection with the interpretation of the description of the subject technology. All structural and functional equivalents to the elements of the various configurations described throughout this disclosure that are known or later come to be known to those of ordinary skill in the art are expressly incorporated herein by reference and intended to be encompassed by the subject technology. Moreover, nothing disclosed herein is intended to be dedicated to the public regardless of whether such disclosure is explicitly recited in the above description.

Although the detailed description contains many specifics, these should not be construed as limiting the scope of the subject technology but merely as illustrating different examples and aspects of the subject technology. It should be appreciated that the scope of the subject technology includes other embodiments not discussed in detail above. Various other modifications, changes and variations may be made in the arrangement, operation and details of the method and apparatus of the subject technology disclosed herein without departing from the scope of the present disclosure. Unless otherwise expressed, reference to an element in the singular is not intended to mean “one and only one” unless explicitly stated, but rather is meant to mean “one or more.” In addition, it is not necessary for a device or method to address every problem that is solvable (or possess every advantage that is achievable) by different embodiments of the disclosure in order to be encompassed within the scope of the disclosure. The use herein of “can” and derivatives thereof shall be understood in the sense of “possibly” or “optionally” as opposed to an affirmative capability.

What is claimed is:

1. A prosthetic heart valve delivery device comprising:
a docking area defining a longitudinal axis; and
an engagement wire extending along the longitudinal axis, the engagement wire comprising a first section, a second section, and a looped section interposed between the first section of the second section along the engagement wire, the engagement wire having (i) an engaged position in which the looped section extends through the docking area and the second section at least partially longitudinally overlaps with the first section to facilitate engagement between the delivery device and a heart valve prosthesis at the docking area and (ii) a disengaged position in which the second section is positioned distal to the looped section and the first section to permit disengagement of the heart valve prosthesis from the docking area.

2. The delivery device of claim 1, further comprising a tubular member having a lumen that at least partially defines the docking area.

3. The delivery device of claim 2, wherein the tubular member comprises at least one aperture extending through a sidewall thereof to the lumen thereof, and wherein in the engaged position, the engagement wire is positioned within the lumen and extends through the aperture.

4. The delivery device of claim 2, wherein the tubular member comprises a distalmost set of apertures extending through a sidewall of the tubular member toward the lumen thereof, the engagement wire extending through the distalmost set of apertures for facilitating engagement between the delivery device and the heart valve prosthesis at the docking area.

5. The delivery device of claim 1, further comprising a tubular member having a lumen that at least partially defines the docking area, the tubular member comprising a plurality of apertures extending from an outer surface of the tubular member to the lumen, the engagement wire extending through the lumen and the plurality of apertures.

6. The delivery device of claim 1, further comprising a tubular member having a lumen that at least partially defines the docking area, an open end that permits access to the docking area, and a plurality of apertures extending from an outer surface of the tubular member to the lumen for permitting the engagement wire to extend out of and into the lumen and across the docking area to engage a portion of the heart valve prosthesis extending into the open end of the tubular member.

7. The delivery device of claim 1, further comprising three tubular members that form respective docking areas for engaging the valve therewith.

8. The delivery device of claim 1, wherein the docking area comprises an enclosure having an opening for receiving a portion of the heart valve prosthesis to permit coupling between the heart valve prosthesis and the delivery device.

9. A prosthetic heart valve delivery device for delivering a heart valve prosthesis, the device comprising:
a core member defining a longitudinal axis;
a proximal sheath extending over the core member; and
a plurality of grasper mechanisms extending along the longitudinal axis within the proximal sheath for engaging the heart valve prosthesis, each grasper mechanism comprising a longitudinal axis, an elongate docking area extending along the longitudinal axis, and an engagement wire extending along the grasper mechanism, the engagement wire comprising a proximal section and a looped section, the looped section extending through the docking area in a direct transverse relative to the longitudinal axis to facilitate engagement between the grasper mechanism and the heart valve prosthesis in an engaged configuration, the engagement wire being proximally retractable to proximally withdraw the looped section from the docking area for disengaging the grasper mechanism from the heart valve prosthesis in a disengaged configuration.

10. The delivery device of claim 9, wherein each grasper mechanism comprises a tubular member wherein the docking area is disposed.

11. The delivery device of claim 10, wherein the tubular member comprises a plurality of apertures extending through a sidewall of the tubular member to a lumen thereof, the plurality of apertures being configured to permit passage of the engagement wire therethrough.

12. The delivery device of claim 11, wherein the plurality of apertures comprises a pair of opposing apertures positioned at approximately the same axial position relative to a longitudinal axis of the tubular member, the opposing apertures being substantially diametrically opposed from each other.

13. The delivery device of claim 10, wherein the tubular member comprises an elongate receptacle disposed along a sidewall of the tubular member, the elongate receptacle extending at least partially into the sidewall, and having an axial length that is greater than a width thereof.

14. The delivery device of claim 13, wherein the receptacle comprises a shoulder area defined by a recess along a length of the receptacle that is configured to receive a portion of the heart valve prosthesis therein, the shoulder area restricting at least one degree of freedom of movement of the portion of the heart valve prosthesis.

15. The delivery device of claim 10, wherein the tubular member comprises proximal apertures extending through a sidewall of the tubular member and an elongate receptacle disposed along a sidewall of the tubular member, wherein in the engaged configuration, the engagement wire extends, in sequence, out of a lumen of the tubular member through a proximal aperture, into the lumen through another proximal aperture, through the receptacle and docking area to a location outside of the lumen, through the proximal apertures to pass through the lumen and to outside the lumen, and into yet another proximal aperture into the lumen.

16. The delivery device of claim 9, wherein each grasper mechanism comprises a tubular member and an anchor tab that has a cross-sectional protrusion or profile configured to engage with a corresponding cross-sectional indentation or profile of the tubular member for restricting relative rotation between the anchor tab and the tubular member when the anchor tab is received within the lumen.

17. The delivery device of claim 10, wherein a distal end portion of the tubular member comprises an anchor slot configured to receive a portion of a valve anchor therein for restricting relative rotation between the valve anchor and the distal end portion about a longitudinal axis of the tubular member.

18. A prosthetic heart valve delivery device comprising:
a grasper mechanism comprising a longitudinal axis, a tubular member having a lumen and an open end, and an engagement wire extending through the lumen toward the open end, the engagement wire having a distal end segment having a protrusion or recess configured to abut a respective recess or protrusion of a clasper tang of a heart valve prosthesis to form a component coupling, wherein:
in an engaged configuration, the distal end segment and the clasper tang longitudinally overlap within the lumen and collectively define a cross-sectional profile that is less than an inner diameter of the lumen such that movement of the distal end segment and the clasper tang is radially constrained by the lumen to restrict movement of the distal end segment relative to the clasper tang, and
the distal end segment of the engagement wire can be moved distally out of the lumen to permit the distal end segment and the clasper tang to become radially displaced from each other to a disengaged configuration to permit the clasper tang to move freely away from the engagement wire for disengaging the heart valve prosthesis from the grasper mechanism.

19. The delivery device of claim 18, wherein the distal end segment of the engagement wire comprises one or more elongate prongs, each elongate prong having an enlarged end portion and a notch portion, and the notch portion being proximal to the enlarged end portion and having a smaller cross-sectional profile than the enlarged end portion.

20. The delivery device of claim 19, wherein each elongate prong comprises a lateral bump, each lateral bump extending radially from a surface of the elongate prong in a direction away from both elongate prongs, the lateral bumps being configured to contact an inner surface of the lumen for maintaining engagement between the distal end segment and the clasper tang of the heart valve prosthesis.
